(19) United States
(12) Patent Application Publication
(10) Pub. No.: US 2024/0157026 A1
(43) Pub. Date: May 16, 2024

(54) METHODS, COMPOSITIONS AND APPARATUS FOR COATING BONE MATERIAL

(71) Applicant: Warsaw Orthopedic, Inc., Warsaw, IN (US)

(72) Inventor: Leenaporn Jongpaiboonkit, Sterling, MA (US)

(73) Assignee: Warsaw Orthopedic, Inc., Warsaw, IN (US)

(21) Appl. No.: 17/988,191

(22) Filed: Nov. 16, 2022

(51) Int. Cl. A61L 27/32; A61L 27/24; A61L 27/34; A61L 27/36

(52) U.S. Cl. CPC A61L 27/32; A61L 27/24; A61L 27/34; A61L 27/3608; A61L 27/365; A61L 2300/252; A61L 2300/404; A61L 2300/414; A61L 2400/12; A61L 2420/02; A61L 2430/02

(57) ABSTRACT

Methods, compositions and apparatus for coating bone material particles (e.g., fibers) are provided. The methods, compositions and apparatus comprise providing a container configured to receive a mineral coating liquid and a plurality of bone material particles (e.g., fibers) therein; adding the plurality of bone material particles (e.g., fibers) to the container; and contacting the plurality of bone material particles (e.g., fibers) with the mineral coating liquid in the container so as to coat each individual bone material particle (e.g., fiber) with the mineral coating liquid to form a plurality of coated bone material particles (e.g., fibers).

### BACKGROUND

Bone defects or bone voids may be caused by several different factors including, but not limited to, trauma, pathological disease, or surgical intervention. Because bone provides both stability and protection to an organism, these defects or voids can be problematic. To address these defects or voids, compositions that contain natural and synthetic materials have been developed. Bone material particles, whether natural bone (e.g., allograft bone) or synthetic bone (e.g., ceramic, polymer) can be used to grow bone in a bone defect. Bone material particles may, depending upon the materials contained within them, be used to repair bone and to impart desirable biological and/or mechanical properties to the bone defect.

A variety of bone repair materials and bone void materials are used in the medical field. Autologous cancellous bone is one type of bone void filler used. This type of bone has the advantage of being both osteoinductive and non-immunogenic. Unfortunately, this type of bone is not frequently available. Moreover, donor site morbidity and trauma add to the limitations of autologous cancellous bone.

Allograft bone is a reasonable bone graft substitute for autologous bone. It is readily available from cadavers and avoids the surgical complications and patient morbidity associated with harvesting autologous bone. Allograft bone is essentially a load-bearing matrix comprising cross-linked collagen, hydroxyapatite, and osteoinductive bone morphogenetic proteins (BMPs). Human allograft bone is widely used in orthopedic surgery. However, allograft bone does not always have the same strength properties or the cells and proteins that can influence the growth of new bone like autograft bone provides. Further, when using allograft bone, there is a slight chance of disease transmission and a reduced effectiveness since bone growth cells and proteins are removed during the cleansing and disinfecting process.

An alternative to autograft and allograft bone is synthetic bone material, such as ceramic based bone material. Hybrid materials composed of organic polymers coated with inorganic minerals have attracted much attention in medicine due to their combination of advantageous properties. Polymeric materials are a desirable base material for biomedical applications, as they can be processed into a variety of sizes and geometries and can be designed to bioresorb or bioabsorb in a controllable timeframe. Therefore, polymeric biomaterials have been featured in a variety of applications including medical devices, tissue engineering scaffolds, and drug delivery systems.

Calcium phosphate based mineral coatings represent desirable surfaces for biomedical applications, as they can be similar in composition to bone tissue and have been shown to promote favorable interactions with natural bone, a property known as “bioactivity”. The surface modification technology associated with mineral coatings seeks to apply an apatite layer with an engineered nanoparticle sized morphology to the surface of a highly porous biphasic calcium phosphate surface. The mineral coated surface has been demonstrated to stimulate bone cells creating an enhanced cellular environment for bone healing. However, as the surface technology accomplishes bone stimulation through nanoparticle sized morphology features, it is difficult to control the application of nanoparticle sized coatings and increase the coating surface area. The coating of bone material particles can often be challenging. For example, when bone material particles are in particulate form, the particles can agglomerate or clump, which often leads to an inferior coating on the bone implant.

Sometimes it is desirable to coat the surface of a bone implant with a nanocoating.

Prior nanocoating of a bone implant has applied the nanocoating to the surface of a bone implant. The nanocoating, however, does not penetrate the bone implant and the nanocoating distributes only on one outermost layer of the bone implant. As the outermost layer degrades over time, the surface of the bone implant may lose its nanocoating partially or completely, and within days, which may reduce integration of the bone implant into the bone defect. Therefore, it would be beneficial to provide an implant including methods, compositions, and/or apparatus for overcoming these challenges.

### SUMMARY

Methods, compositions, and apparatus are provided for coating bone material particles with a mineral coating to form mineral coated bone material particles. These mineral coated bone material particles can be formed into a bone implant that can have the coated bone material particles integrally formed in it and/or homogenously distributed throughout the bone implant. The coated bone material particles have a nanocoating and/or a nanocoating structure with a particular dimension and geometry (e.g., a plate like morphology) to increase the surface area, which enhances bioactivity of the bone material particles and is beneficial in bone remodeling.

In some embodiments, there is a coated bone material particle, the coated bone material particle having a mineral coating thereon, the mineral coating having nanostructures and/or a thickness of 1000 nanometers or less, the mineral coating comprising hydroxyapatite.

In some embodiments, a coated bone material particle is provided, the coated particle comprising demineralized bone, cellular bone material, collagen or a combination thereof, the coated particle having a mineral coating thereon, the mineral coating comprising nanostructures having an average size range from 1 nanometer to less than 1 micron or from about 5 to about 500 nanometers, wherein the coated particle has a BET surface area from about 0.1 to about 9.5 m²/g.

In some embodiments, a coated fiber is provided, the coated fiber comprising demineralized bone, cellular bone material, collagen or a combination thereof, the coated fiber having a mineral coating thereon, the mineral coating comprising nanostructures having an average size range from 1 nanometer to less than 1 micron or from about 5 to about 500 nanometers, wherein the coated fiber has a BET surface area from about 0.1 to about 9.5 m²/g.

In some embodiments, a method of coating bone material fibers is provided. The method comprises providing a container configured to receive a mineral coating liquid and a plurality of bone material fibers therein; adding the plurality of bone material fibers to the container; and contacting the plurality of bone material fibers with the mineral coating liquid in the container so as to coat each individual bone material fiber with the mineral coating liquid to form a plurality of coated bone material fibers.

In some embodiments, an apparatus for coating bone material fibers is provided. The apparatus comprises a first mesh having a first set of openings configured to allow a mineral coating liquid and a plurality of bone material fibers of a select size therethrough and a plurality of bone material fibers larger than the select size to remain on the first mesh; and a housing or container partially enclosing at least the first mesh, the housing or container having an outlet configured to be fluidly coupled to a pump, the pump configured to provide the coating liquid to at least the first mesh to coat the plurality of bone material fibers.

While multiple embodiments are disclosed, still other embodiments of the present application will become apparent to those skilled in the art from the following detailed description, which is to be read in connection with the accompanying drawings. As will be apparent, the present disclosure is capable of modifications in various obvious aspects, all without departing from the spirit and scope of the present disclosure. Accordingly, the detailed description is to be regarded as illustrative in nature and not restrictive.

### BRIEF DESCRIPTION OF THE FIGURES

In part, other aspects, features, benefits, and advantages of the embodiments will be apparent regarding the following description, appended claims and accompanying drawings.

FIG. 1 illustrates uncoated bone material fibers. The bone material fiber on the left illustrates an uncoated collagen fiber, which is shown in a coiled configuration for simplicity. The bone material fiber on the right illustrates an uncoated Demineralized Bone Matrix (DBM) fiber, which is shown in a linear configuration for simplicity. However, it will be understood that the fibers can have any configuration and can be entirely coiled, uncoiled, or linear or have portions that are coiled, uncoiled, or linear.

FIG. 2 illustrates one embodiment of the coating method where a plurality of individual collagen fibers are disposed in a container and contacted with a mineral coating liquid to form a mineral coating on each individual collagen fiber. After removal from the container, the mineral coating is uniformly distributed on multiple surfaces of each individual collagen fiber.

FIG. 3 illustrates one embodiment of the coating method where a plurality of individual DBM fibers are disposed in a container and contacted with a mineral coating liquid to form a mineral coating on each individual DBM fiber. The mineral coating is uniformly distributed on multiple surfaces of each individual DBM fiber as shown in FIG. 2.

FIG. 4 is a side view of one embodiment of an apparatus of the current application that illustrates the coating of bone material fibers within a first (top) mesh and second (bottom) mesh shown in a stacked configuration. The apparatus comprises a pump fluidly coupled to a container of coating liquid (e.g., mineral coating liquid) that directs coating liquid to the first mesh and provides a coating bath to coat larger bone material fibers (e.g., collagen fibers) in the first mesh and smaller bone material fibers (e.g., collagen fibers) in the second mesh. Excess coating liquid is recycled or replenished in this embodiment.

FIG. 5 is a side view of one embodiment of an apparatus of the current application that illustrates the coating of bone material fibers within a first (top) mesh and second (bottom) mesh shown in a stacked configuration. The apparatus comprises a pump fluidly coupled to a container of coating liquid (e.g., mineral coating liquid) that directs coating liquid to the first mesh and provides a coating bath to coat larger bone material fibers (e.g., DBM fibers) in the first mesh and smaller bone material fibers (e.g., DBM fibers) in the second mesh. In this embodiment, both meshes are submerged in a container. Excess coating liquid is also recycled or replenished in this embodiment.

FIG. 6 is a flow diagram illustrating an embodiment of the coating method. The method includes providing bone material fibers; coating bone material fibers in nanocoating bath containing a mineral coating liquid; drying nanocoated bone material fibers; then placing dried nanocoated bone material fibers in a mold of a desired shape; optionally lyophilizing the nanocoated bone material fibers that are in the mold; removing the nanocoated bone material fibers from the mold; and packaging the molded, nanocoated bone material fibers for a ready-to-use bone implant. The nanocoated bone material can have a mineral coating thereon with a thickness of up to 5 microns.

FIG. 7 is a perspective view of a plurality of dried, coated collagen fibers. The dried fibers are shown in this particular embodiment loosely entangled together.

FIG. 8 is a perspective view of a plurality of dried, coated DBM fibers. The fibers are shown in this particular embodiment clumped together.

FIG. 9 is a perspective view of a plurality of dried, coated collagen fibers that are removed from the mold. The coated collagen fibers are lyophilized into a bone implant having various molded shapes. The shapes shown include a cube, a sphere and a pyramid. It will be understood that the bone implant can be any shape.

FIG. 10 is a perspective view of a plurality of dried, coated DBM fibers that are removed from the mold. The coated DBM fibers are lyophilized into an implant having various molded shapes. The shapes include a cube, a rectangle and a disc. It will be understood that the bone implant can be any shape.

It is to be understood that the figures are not drawn to scale. Further, the relation between objects in a figure may not be to scale and may in fact have a reverse relationship as to size. The figures are intended to bring understanding and clarity to the structure of each object shown, and thus, some features may be exaggerated to illustrate a specific feature of a structure.

### DETAILED DESCRIPTION

### Definitions

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Similarly, when values are expressed as approximations, by use of the antecedent “about,” it will be understood that the particular value forms another embodiment that is +/−10% of the recited value. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value.

Notwithstanding that the numerical ranges and parameters setting forth the broad scope of this application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

“Biocompatible”, as used herein, is intended to describe materials that, upon administration in vivo, do not induce undesirable long-term effects.

“Bone”, as used herein, refers to bone that is cortical, cancellous, or cortico-cancellous of autogenous, allogenic, xenogenic, or transgenic origin.

“Bone material particles” include natural or synthetic granules, cellular matrix, segments, fibers, powder, chips, shards, demineralized bone particles, surface demineralized bone particles, ceramic particles, collagen granules, collagen fibers, collagen powder, collagen shards, collagen particles or a combination thereof. Bone material particles, whether natural or synthetic can be in any shape including triangular, oval, oblong, spherical, cube shaped, cylindrical shape, disc shaped, or other shapes having regular, irregular or random geometries.

“Bone material fibers” include bone material sourced from natural bone or synthetic bone in a fibrous form having a particular ratio of the longest average dimension of the fiber (average length) to its shortest average dimension (average thickness). This is also referred to as the “aspect ratio” of the fiber. Bone material fibers, as used herein, include bone elements whose average length to average thickness ratio or aspect ratio of the fiber is from about 50:1 to about 1000:1. In some embodiments, average length to average thickness ratio or aspect ratio of the fiber is from about 50:1, 75:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1 and/or 1000:1. Any aspect ratio that provides a linear configuration of the bone particle would be characterized as a segment or fiber. In overall appearance the bone material fibers can be described as bone fibers, threads, narrow strips, or thin sheets. Often, where thin sheets are produced, their edges tend to curl up toward each other. The bone material fibers can be substantially linear in appearance or they can be coiled to resemble springs. In some embodiments, the entire bone material fiber can be linear or coiled or have portions that are linear and coiled. In some embodiments, the bone material fibers are of irregular shapes including, for example, serpentine or curved shapes. In some embodiments, the bone material fibers comprise collagen. In some embodiments, the bone material fibers can be mineralized. In some embodiments, the fibers are a combination of demineralized and mineralized fibers. In some embodiments, the bone material fiber comprises DBM fiber. In some embodiments, the bone material fibers include demineralized bone matrix (DBM) fibers, collagen fibers or a combination thereof.

“Demineralized bone matrix (DBM),” as used herein, refers to any material generated by removing mineral material from bone tissue. In some embodiments, the DBM fibers as used herein include preparations containing less than 5% calcium and, in some embodiments, less than 1% calcium by weight. In other embodiments, the DBM fibers comprise partially demineralized bone (e.g., preparations with greater than 5% calcium by weight but containing less than 100% of the original starting amount of calcium).

“Collagen”, as used herein includes, but is not limited to, pure or native collagen material or collagen material with other extracellular matrix constituents (e.g., proteins such as noncollagenous proteins, including growth factors, bone morphogenic proteins (BMPs), etc.). Any suitable collagen material may be used in the current application for the collagen fiber. Suitable collagen material for the collagen fiber is described in U.S. Publication No. 2008/0260794A1, assigned to Warsaw Orthopedics, which is hereby incorporated by reference in its entirety.

Bone material that is “non-fibrous” or not in fiber form includes bone material that is in powder, granule, oval, sphere, cube, cylinder, or triangular shape.

The term “autograft” refers to graft material harvested from the same individual patient who is also recipient of the graft, obtained surgically from non-essential donation sites in the patient.

Bone graft, as used herein, refers to any implant prepared in accordance with the embodiments described herein and therefore may include expressions such as a bone void filler.

The term “nano-sized feature” includes recesses, projections or a combination thereof that are in nanometer size.

The term “osteoinductive,” as used herein, refers to the quality of being able to recruit cells from the host that have the potential to stimulate new bone formation. Any material that can induce the formation of ectopic bone in the soft tissue of an animal is considered osteoinductive.

The term “osteoinduction” refers to the ability to stimulate the proliferation and differentiation of pluripotent mesenchymal stem cells (MSCs). In endochondral bone formation, stem cells differentiate into chondroblasts and chondrocytes, laying down a cartilaginous ECM, which subsequently calcifies and is remodeled into lamellar bone. In intramembranous bone formation, the stem cells differentiate directly into osteoblasts, which form bone through direct mechanisms. Osteoinduction can be stimulated by osteogenic growth factors, although some ECM proteins can also drive progenitor cells toward the osteogenic phenotype.

The term “osteogenic” refers to the ability of a graft material to produce bone independently. To have direct osteogenic activity, the graft must contain cellular components that directly induce bone formation. For example, an allograft seeded with activated MSCs would have the potential to induce bone formation directly, without recruitment and activation of host MSC populations. Because many osteoconductive allografts also can bind and deliver bioactive molecules, their osteoinductive potential will be greatly enhanced.

The term “patient” refers to a biological system to which a treatment can be administered. A biological system can include, for example, an individual cell, a set of cells (e.g., a cell culture), an organ, or a tissue. Additionally, the term “patient” can refer to animals including, but not limited to humans, other primates such as chimpanzees, apes, orangutans and monkeys, rats, mice, cats, dogs, cows, horses, etc.

The term “implantable” as used herein refers to a biocompatible device (e.g., the bone material fibers) retaining potential for successful placement within a mammal. The expression “bone implant” and expressions of the like as used herein refer to an object implantable through surgery, injection, or other suitable means whose primary function is achieved either through its physical presence or mechanical properties.

The term “moldable” includes that the bone implant can be shaped by hand or machine or injected into the target tissue site (e.g., bone defect, fracture, or void) into a wide variety of configurations to fit within the bone defect. In some embodiments, the bone implant including the mineral coated fibers can be placed into a mold and molded into the desired shape.

The term “cohesive” as used herein means that the composition tends to remain a singular, connected mass upon the addition of fluid, autograft bone or during manipulation, including the exhibition of the ability to be molded or shaped without breaking upon manipulating, or disintegrating or becoming unstable.

The term “flowable” includes that when the bone material particles (e.g., fibers) are mixed with a fluid, they can be administered in an injectable state via a syringe and/or cannula. The bone material particles (e.g., fibers) can be flowable when its consistency is fluid-like and has a viscosity that is lower than that of the viscosity of the bone material particles (e.g., fibers) when in a putty or paste form. Flowable bone material particles (e.g., fibers) include liquid or fluid (e.g., solution, suspension, or the like) or semi-solid compositions (e.g., gels, cements) that are easy to manipulate and may be brushed, sprayed, dripped, injected, shaped and/or molded at or near the target tissue site. In various embodiments, the mineral coated bone material particles (e.g., fibers) may be used to fill one or more voids in a bone defect (e.g., an osteolytic lesion).

The term “hydrate,” “hydration,” “hydratable,” “hydrating’ or “hydrated” refers to adding an amount of fluid to the coated bone material particles (e.g., fibers) to increase the amount of moisture content in the composition to form a gel, putty or paste that is flowable.

The term “dehydrated” or “dehydration” refers to bone material particles (e.g., fibers) that contain a small amount of residual moisture or no moisture content and can be in the form of a dry composition. The dehydrated composition can have a moisture content from about 0 to about 10% based on the total weight of the composition. In some embodiments, when a composition is dehydrated, fluid can be added to the composition to hydrate the composition. A dehydrated composition includes a lyophilized or freeze-dried composition.

The term “lyophilized” or “freeze-dried” includes a state of a substance that has been subjected to a drying procedure such as lyophilization, where at least 50% of moisture has been removed. The bone material particles (e.g., fibers) may be lyophilized or freeze-dried before and after molding.

The term “bone marrow aspirate” or “BMA” refers to bone marrow fluid that can be obtained via a syringe and needle to harvest the bone marrow fluid from the patient. Bone marrow aspirate comprises fluid that contains a heterogeneous mix of stem and progenitor cells, platelets, and white blood cells. The bone marrow aspirate can be harvested from various sources in the body including, but not limited to, the iliac crest. In some embodiments, the BMA can be used to hydrate the bone material fibers.

The headings below are not meant to limit the disclosure in any way; embodiments under any one heading may be used in conjunction with embodiments under any other heading.

### Coating Methods and Apparatus

Methods, compositions, and apparatus are provided for coating bone material particles with a mineral coating to form mineral coated bone material particles. These mineral coated bone material particles can be formed into a bone implant that can have the coated bone material particles integrally formed in it and/or homogenously distributed throughout the bone implant. The coated bone material particles have a nanocoating and/or a nanocoating structure with a particular dimension and geometry (e.g., a plate like morphology) to increase the surface area, which enhances bioactivity of the bone material particles and is beneficial in bone remodeling.

Methods and apparatus are provided for coating bone implants with a mineral coating that can be efficiently applied to each bone material fiber, which can be used to form the bone implant. The methods and apparatus of the current application utilize a container system allowing each individual bone material fiber to have sufficient contact with the mineral coating. In one embodiment, a container is used to hold a plurality of bone material fibers. The fibers are loosely disposed inside the container to maximize contact surfaces between the fibers and the coating solution. In another embodiment, a mesh system is used with at least one mesh that can be stacked with one or more meshes. The meshes have a plurality of openings for smaller bone material fibers and coating liquid (e.g., mineral coating) to pass therethrough, while larger bone material fibers that are larger than the openings stay on top of the mesh. Typically, meshes with larger openings are stacked on top of meshes with smaller openings. In this way, bone material fibers of select sizes can be contacted (e.g., incubated, submerged, suspended, bathed, etc.) with coating liquid. These bone material fibers can not only have an increased coating surface applied to their surfaces, but the coating application to the bone material fibers can carefully be controlled.

The current application allows increased surface exposure of the coating liquid to the surface of the bone material fibers, which in turn allows increased sporadic mineral nucleation on the surface of the bone material fibers. These fibers are coated individually instead of being coated as a massive clump or instead of the entire formed implant being coated all at once. The individual bone material fibers that are coated individually with the mineral coating have multiple surfaces of the fiber coated with the mineral coating. These coated individual bone material fibers can be formed into the bone implant that now has increased surface area, which enhances bioactivity of the bone material fibers and is beneficial in bone remodeling.

In some embodiments, the bone material fibers can be collagen fibers, DBM fibers or a combination thereof. The fibers can be coated individually with the mineral coating to ensure similar geometry and characteristics to enhance the contact surface area and the integrity of coating.

In some embodiments, in the coating process, optionally meshes can be placed in a container and used in the coating method. The meshes can have openings of the same size or each mesh can have openings of a different size. In some embodiments, a mesh can be stacked that does not have bone material fibers thereon and will allow coating liquid to pass through its openings.

In some embodiments, when different size meshes are used, the different size meshes also allow bone material fibers of different sizes to be isolated on meshes that have smaller openings than the bone material fibers. This allows reduction in bone material fibers wastage as the bone material fibers can be captured on the mesh. The meshes also allow spacing of the bone material fibers and reduce the chance of the bone material fibers hitting each other, which can disrupt the integrity of the coating.

In some embodiments, the coated bone material fibers can be lyophilized and coated again before and after molding to ensure the distribution of the coating materials. The coated bone material fibers can be dried before lyophilization. After the bone material fibers are coated, the coated bone material fibers can be dried, lyophilized and then molded into various shapes including triangular, oval, oblong, spherical, cube-shaped, cylindrical, disc-shaped, or other shapes having regular, irregular or random geometries. The molded, coated bone material fibers can be used, individually or in combination, to form a coated bone implant. The coated bone implant can be further coated or can be used directly in a patient.

In some embodiments, the dried fibers can be mixed with a binder e.g., uncoated collagen gel or glycerol, which will not require a lyophilizing step.

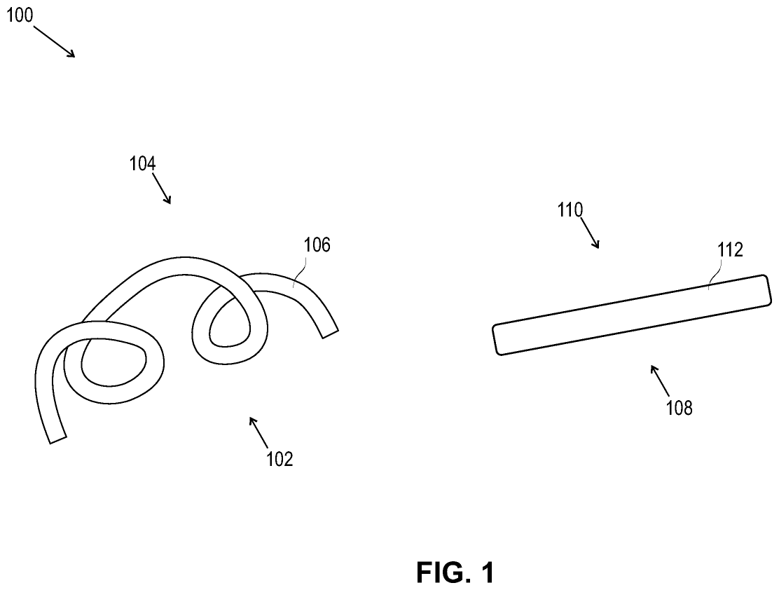

FIG. 1 illustrates an embodiment of uncoated bone material fibers 100. Bone material fibers can comprise a collagen fiber 102, a DBM fiber 108, or a combination thereof. The uncoated collagen fiber 104 generally has a coiled pattern 106 and the uncoated DBM fiber 110 generally has a linear pattern 112. In various embodiments, both the collagen fibers and the DBM fibers may have various geometries. Under different magnifications and viewing angles, collagen fibers may appear to be linear while DBM fibers may appear to have curves. However, it will be understood that the fibers can have any configuration and can be entirely coiled, uncoiled, or linear or have portions that are coiled, uncoiled, or linear.

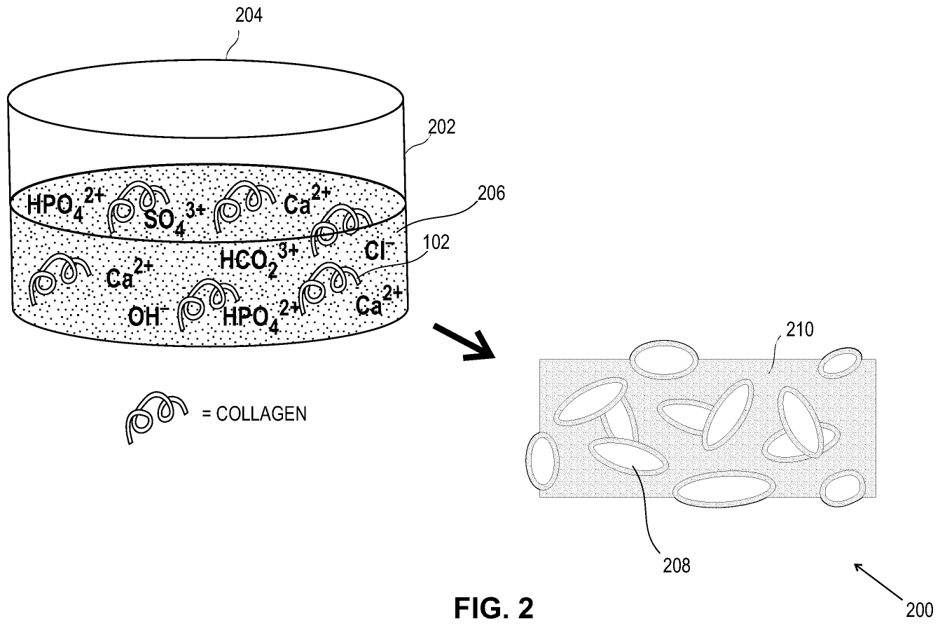

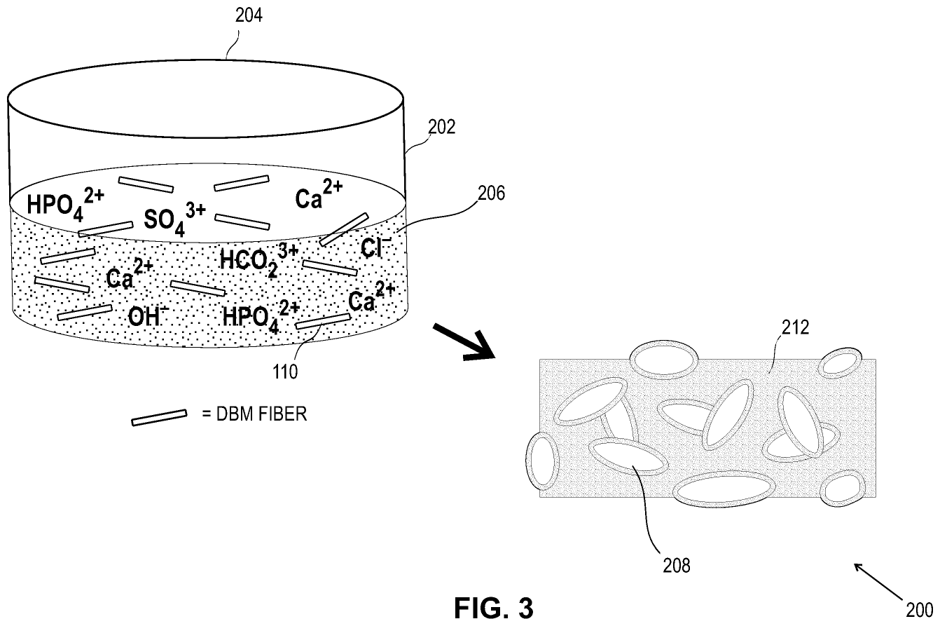

FIGS. 2 and 3 illustrate an embodiment of the individual coated bone material fiber 200 in a container system. In FIG. 2, uncoated collagen fibers 102 are placed in coating liquid 206 inside container 202 through container opening 204 and are subjected to a mineral coating liquid bath. In FIG. 3, uncoated DBM fibers 110 are placed in a coating liquid 206 (e.g., a mineral coating liquid) inside container 202 through container opening 204. In some embodiments, a container comprises a beaker, bag, flask, tray, cup, glass, mug, or tumbler capable of holding coating liquid and allows easy retrieval of coated fibers from the container. In some embodiments, uncoated collagen fibers are distributed loosely to avoid clumping, and this ensures that each individual fiber has multiple surfaces contacted with the coating liquid (e.g., a mineral coating liquid). Once removed from the coating liquid, a coated collagen fiber 210 has a mineral coating 208 uniformly distributed on multiple surfaces of the coated collagen fiber. In some embodiments, once removed from the coating liquid, a coated DBM fiber 212 has a mineral coating 208 uniformly distributed on multiple surfaces of the coated DBM fiber. In some embodiments, the mineral coating liquid is uniformly disposed on a surface of each individual bone material fiber (e.g., collagen fiber, or DBM fiber). In some embodiments, the mineral coating liquid is uniformly disposed on all surfaces of each individual bone material fiber (e.g., collagen fiber, or DBM fiber).

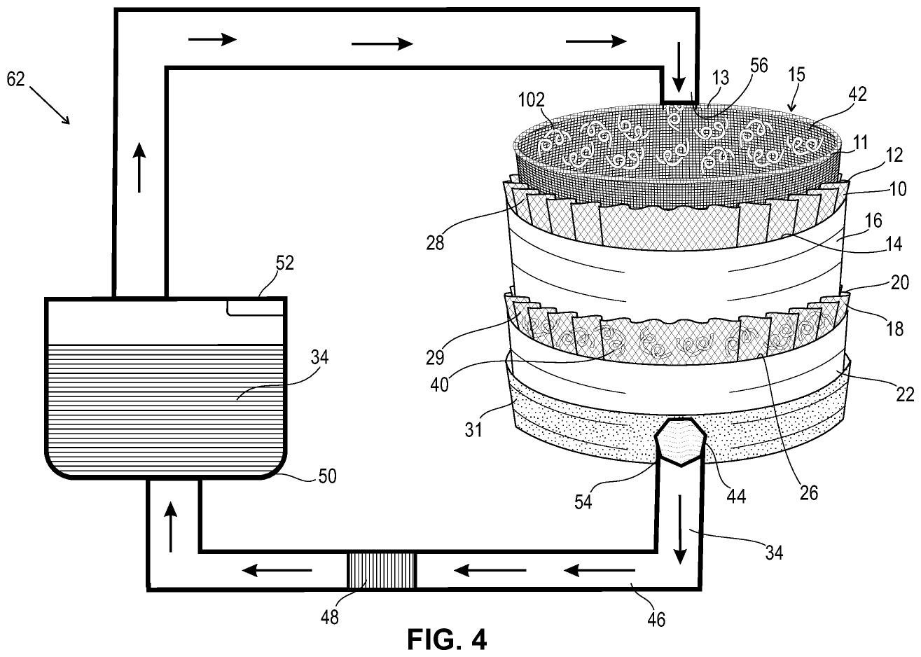

Referring to FIG. 4, an apparatus for coating bone material fibers is provided using at least two meshes that allow coating of bone material fibers on each layer of the mesh. The apparatus 62 comprises a plurality of stackable meshes (e.g., a first mesh 10 and a second mesh 18). The first mesh has housing 16 and the second mesh has second housing 22 and the meshes are shown in a stacked configuration. It will be understood by one of ordinary skill in the art that the housing that surrounds each mesh is optional.

Bone material fibers of various sizes are added to the top portion of the first mesh 10. Large bone material fibers 42 are caught and disposed on the bottom portion 14 of the first mesh. Small bone material fibers 40 and coating liquid 34 (e.g., mineral coating liquid) pass through the first set of mesh openings 28 at the bottom portion 14 of the first mesh and flow into the top portion 20 of the second mesh. Small bone material fibers then are disposed on the bottom portion 26 of the second mesh. The bottom portion of the first mesh has a first set of openings 28 that allows smaller bone material fibers and coating liquid to slowly drain into and be captured by the second mesh (not shown). In this way, the bone material fibers can have more coated surface(s) by controlled addition of the coating liquid contacting the bone material fibers causing each individual bone material fiber to be isolated, submerged, suspended, bathed, and/or incubated in the coating liquid in the top portion 12 within the housing 16 of the first mesh 10. It will be understood that a plurality of meshes (not shown) can be stacked below the first mesh, including for example, two, three, four, five, six, seven, eight, nine, ten, eleven, twelve, thirteen, fourteen, fifteen, sixteen, seventeen, eighteen, or more. In some embodiments, the term “above” is referred to a geographical location at a higher level in altitude on a vertical axis. In some embodiments, the term “below” is referred to a geographical location at a lower level in altitude on a vertical axis. Typically, the meshes are stacked on one another substantially parallel to each other.

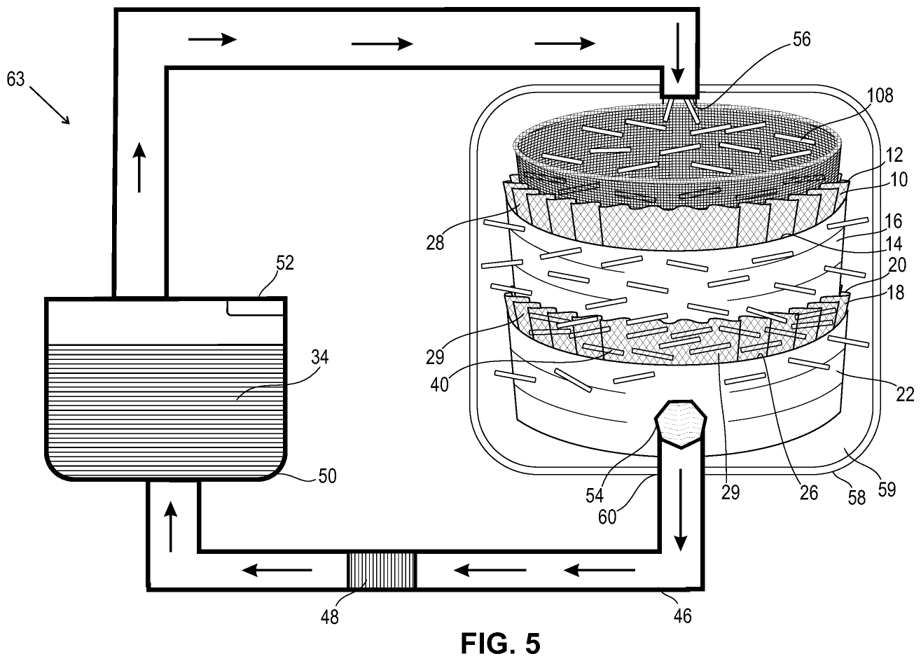

Shown in FIGS. 4 and 5, the bone material fibers are spaced apart on the mesh and the mesh allows less bone material fiber interaction with each other and maximizes the bone material fibers' surface area with the coating liquid. This allows a uniform incubation and coating of the bone material fibers to form coated bone material fibers 36 on one layer. It will be understood that the bone material fibers and coating liquid can be added to the mesh in any order. Therefore, the bone material fibers can be added to the mesh first and then the coating liquid, or the coating liquid can be added to the mesh first and then the bone material fibers or both the coating liquid and the bone material fibers can be added to the mesh simultaneously. In some embodiments, the coating liquid and the bone material fibers can be mixed together and then added to the mesh.

The bone material fibers can have various sizes. The sizes of the large bone material fibers and the small bone material fibers are relative to the batch of uncoated bone material fibers to be used for coating and/or the sizes of the mesh pores. For example, large bone material fibers having a size greater than 255 nanometers (nms) will stay in a layer on the first mesh. The first mesh can have a first set of openings less than 255 nms. Bone material fibers having a size smaller than 255 nms will flow through the first set of openings 28 of the first mesh and flow into the second mesh 18. The smaller bone material fibers, which have a size smaller than 255 nms but larger than 212 nms will stay in a layer of the second mesh. Bone material fibers with sizes smaller than 212 nms and coating liquid will flow through the second set of mesh openings 29 as they are smaller than the second set of mesh openings and flow into the layer of the third mesh (not shown).

In some embodiments, the bone material fibers with sizes between 212 nms and 160 nms, and all remaining bone material fibers will be captured and stay on the third mesh in one layer. In some embodiments, the bone material fibers with sizes between 212 nms and 160 nms will stay on the layer of the third mesh and all the remaining bone material fibers and coating liquid will pass through the third mesh and flow into a collection container. In some embodiments, whether the remaining bone material fibers pass through the bottommost layer of the stackable mesh depends on the distribution of bone material fibers in each layer so maximum spacing between each bone material fiber can be achieved to prevent interference of the fiber interactions during mineralization of the coating liquid on the surface of the bone material fibers.

In some embodiments, the apparatus further comprises a collection container 31 under or directly below the bottommost mesh (e.g., the second mesh) such that the second set of openings 29 at the bottom of the second mesh allows smaller bone material fibers and coating liquid to flow through the second set of openings 29 of the second mesh. The collection container 31 can capture excess coating liquid and/or small bone material fibers, where they can be recirculated back to the meshes.

In some embodiments, the coating liquid can be added to the stackable meshes to a fill line, which indicates the level of coating liquid that can contact the bone material fibers so that they can be incubated, submerged, suspended, and/or bathed with coating liquid. The meshes allow the bone material fibers to be spread out or distributed loosely away from each other so that each bone material fiber has a greater surface area exposed to coating liquid.

In the apparatus 62 shown in FIG. 4, the second housing 22 or the collection container 31 can comprise an outlet 44 that allows drainage of the coating liquid. In the embodiment shown, the outlet 44 is disposed on a sidewall of a housing (e.g., the second housing 22) or a sidewall of the collection container 31. In some embodiments, the outlet comprises a filter (not shown) that prevents the bone material fibers from flowing out of the stackable meshes.

The apparatus 62 can also comprise a tube 46 fluidly coupled to the outlet 44 at tube inlet 54 that leads to fluid pump 48, which pumps collected coating liquid to tube outlet 56, which delivers the coating liquid back to the stackable meshes. The fluid pump assists in pumping the fluid from the tube inlet through the tube outlet. In this way, collected coating liquid can be recirculated back to the meshes for coating the bone material fibers.

The apparatus 62 can also comprise a container 50 for storing coating liquid. The container 50 has port 52 that is configured to receive additional coating liquid or replenish coating liquid as illustrated in FIG. 4.

Referring to FIG. 5, the second apparatus 63 is similar to the apparatus 62 in FIG. 4, except in the embodiment of FIG. 5, a second container 58 is provided. The container is connected to the tube and capable of replenishing additional coating liquid (e.g., mineral coating liquid) to the stackable meshes. In some embodiments, the apparatus further comprises a second container 58. The second container 58 is configured to hold and partially enclose the stackable meshes. The second container 58 can comprise an outlet 60 of the second container. The outlet 60 of the second container can be disposed on a sidewall 59 of the second container. The outlet 60 can be aligned with outlet 44 (not shown) of the second housing 22. This allows the outlet to drain coating liquid to tube 46, which is fluidly coupled to pump 48, which pumps coating liquid out to outlet 56. The apparatus allows coating liquid to be recirculated or replenished. In the embodiment shown in FIG. 5, the first mesh and the second mesh are immersed in coating liquid in the second container. In some embodiments, the container is configured to enclose at least the first mesh, at least the second mesh, or at least the first and the second mesh.

In some embodiments, the methods and apparatus of the current application can be conducted continuously through continuous pumping and draining of the coating liquid from the second container and/or the bottommost housing of the stackable mesh. In some embodiments, the method can be conducted in a batch process such that coating liquid is all drained before new and/or unused coating liquid is added back into the second container or into the first mesh. In some embodiments, the apparatus can comprise a weight 11 on top and/or above the first mesh to keep the first mesh from overflowing or moving in the second container. The weight can be in various shapes that do not interfere with the additions of the coating liquid and/or bone material fibers. In some embodiments, the top portion 20 of the second mesh can have an area where bone material fibers and coating liquid can be introduced.

After the bone material fibers are coated, the meshes can be removed and the meshes containing the bone material fibers can be placed directly into the oven for drying without transferring the coated bone material fibers from the mesh.

It will be understood that, in some embodiments, in the coating process, a mesh is optional and can have the same size openings as the mesh stacked above or below it. The same size mesh can be stacked immediately adjacent to a mesh having the same size openings or stacked intermittently among different layers of the meshes having different size openings. For example, a plurality of meshes having the same size openings can be the first mesh and these meshes can be stacked immediately adjacent to each other or distributed above or below meshes having a smaller size opening or a larger size opening to form a pattern. In some embodiments, the stackable meshes comprise a cover mesh 13 having openings 15 to allow coating liquid with or without bone material fibers therethrough. The cover mesh can be stacked on top of the first mesh or can be stacked on top of all the other meshes in the stack. In some embodiments, the cover mesh can have no bone material fibers on it, and the cover mesh can be used to ensure that coating liquid flow is the same to meshes stacked below the cover mesh.

It will be understood by those of ordinary skill in the art that meshes can be stacked above or below each other but do not need to directly contact each other. Therefore, the meshes can be spaced a distance between each other that allows optimal flow of coating liquid and material fibers from one mesh to the other. In the embodiment, the housing 16 of the first mesh and the second housing 22 of the second mesh allow the optimal distance for the flow of bone material fibers and coating liquid. In some embodiments, the first mesh can be spaced parallel to the second mesh, where they are both fluidly communicating with each other.

After the coating process, the coated bone material fibers are transferred to a drying area (e.g., oven). In some embodiments, the method comprises drying the coated bone material fibers remaining on the optional first mesh, optional second mesh, optional third mesh or all of the optional meshes in an oven.

### Bone Material Particles

Bone material particles that can have the liquid mineral coating applied thereto include natural bone, synthetic bone, and/or collagen in particulate form. Bone material particles include natural or synthetic granules, fibers, powder, chips, shards, demineralized bone particles, surface demineralized bone particles, ceramic particles, collagen granules, collagen fibers, collagen powder, collagen shards, collagen particles or a combination thereof. Bone material particles, whether natural or synthetic can be in any shape including triangular, oval, oblong, spherical, cube shaped, cylindrical shape, disc shaped, or other shapes having regular, irregular or random geometries.

Bone material particles of particular interest include natural bone or synthetic bone in particulate form and can be coated with a polymer (e.g., collagen) before being coated with the liquid coating. Bone material fibers include natural or synthetic demineralized bone material fibers, surface demineralized bone material fibers, fibrous connective tissue, fibrous collagen tissue, fibrous bone material or a combination thereof. Bone material fibers, whether natural or synthetic can be made in any shape including triangular, oval, oblong, spherical, cube-shaped, cylindrical, disc-shaped, or other shapes having regular, irregular or random geometries. In particular, the bone material fibers include collagen fibers and demineralized bone matrix (DBM) fibers as discussed herein.

In some embodiments, the bone material fibers that can be added for coating can be an inorganic material in fibrous form having a certain aspect ratio, such as an inorganic ceramic and/or bone substitute material. Exemplary inorganic materials or bone substitute materials include, but are not limited to, aragonite, dahlite, calcite, brushite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™ fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, the bone material fibers can be mixed with other suitable bone materials used for bone implantable compositions that can be added for coating. Other suitable bone materials can comprise mineral particles in fibrous form having a certain aspect ratio, which for exemplary purposes and not limitation here, comprise tricalcium phosphate and hydroxyapatite in a ratio of about 80:20 to about 90:10. In some embodiments, the mineral particles can comprise tricalcium phosphate and hydroxyapatite in a ratio of about 70:30 to about 95:5. In some embodiments, the mineral particles can comprise tricalcium phosphate and hydroxyapatite in a ratio of about 85:15.

Following shaving, milling or other technique whereby they are obtained, the bone material, in some embodiments, is subjected to demineralization in order to reduce its inorganic content to a very low level, in some embodiments, to not more than about 5% by weight of residual calcium and preferably to not more than about 1% by weight residual calcium. Demineralization of the bone material ordinarily results in its contraction to some extent.

Bone used in the methods described herein may be autograft, aliograft, or xenograft. In various embodiments, the bone may be cortical bone, cancellous bone, or cortico-cancellous bone. While specific discussion is made herein to demineralized bone matrix, bone matrix treated in accordance with the teachings herein may be non-demineralized, demineralized, partially demineralized, or surface demnineralized. The following discussion applies to demineralized, partially demineralized, and surface demineralized bone matrix. In one embodiment, the demineralized bone is sourced from bovine or human bone. In another embodiment, demineralized bone is sourced from human bone. In one embodiment, the demineralized bone is sourced from the patient's own bone (autogenous bone). In another embodiment, the demineralized bone is sourced from a different animal (including a cadaver) of the same species (allograft bone).

Any suitable manner of demineralizing the bone may be used. Demineralization of the bone material can be conducted in accordance with known conventional procedures. For example, in a preferred demineralization procedure, the bone materials useful for the implantable composition of this application are subjected to an acid demineralization step that is followed by a defatting/disinfecting step. The bone material can be immersed in acid over time to affect its demineralization. Acids which can be employed in this step include inorganic acids such as hydrochloric acid and organic acids such as peracetic acid, acetic acid, citric acid, or propionic acid. The depth of demineralization into the bone surface can be controlled by adjusting the treatment time, temperature of the demineralizing solution, concentration of the demineralizing solution, agitation intensity during treatment, and other applied forces such as vacuum, centrifuge, pressure, and other factors such as known to those skilled in the art. Thus, in various embodiments, the bone material may be fully demineralized, partially demineralized, or surface demineralized.

After acid treatment, the bone is rinsed with sterile water for injection, buffered with a buffering agent to a final predetermined pH and then finally rinsed with water for injection to remove residual amounts of acid and buffering agent or washed with water to remove residual acid and thereby raise the pH. Following demineralization, the bone material is immersed in solution to effect its defatting. A preferred defatting/disinfectant solution is an aqueous solution of ethanol, the ethanol being a good solvent for lipids and the water being a good hydrophilic carrier to enable the solution to penetrate more deeply into the bone. The aqueous ethanol solution also disinfects the bone by killing vegetative microorganisms and viruses. Ordinarily at least about 10 to 40 weight percent by weight of water (i.e., about 60 to 90 weight percent of defatting agent such as alcohol) should be present in the defatting/disinfecting solution to produce optimal lipid removal and disinfection within the shortest period of time. The preferred concentration range of the defatting solution is from about 60 to 85 weight percent alcohol and most preferably about 70 weight percent alcohol.

Further in accordance with this application, the DBM material can be used immediately for preparation of the implant or it can be stored under aseptic conditions, advantageously in a critical point dried state prior to such preparation. In a preferred embodiment, the bone material can retain some of its original mineral content such that the composition is rendered capable of being imaged utilizing radiographic techniques.

In various embodiments, this application also provides bone matrix compositions comprising critical point drying (CPD) fibers. DBM may include bone morphogenic proteins (BMPs) and other growth factors. It can be made into fibers and formulated for use as granules, gels, sponge material or a putty and can be freeze-dried for storage. Sterilization procedures used to protect from disease transmission may reduce the activity of beneficial growth factors in the DBM. DBM provides an initial osteoconductive matrix and exhibits a degree of osteoinductive potential, inducing the infiltration and differentiation of osteoprogenitor cells from the surrounding tissues.

DBM preparations have been used for many years in orthopedic medicine to promote the formation of bone. For example, DBM has found use in the repair of fractures, in the fusion of vertebrae, in joint replacement surgery, and in treating bone destruction due to underlying disease such as rheumatoid arthritis. DBM is thought to promote bone formation in vivo by osteoconductive and osteoinductive processes. The osteoinductive effect of implanted DBM compositions is thought to result from the presence of active growth factors present on the isolated collagen-based matrix. These human growth factors include members of the TGF-β, IGF, and BMP protein families. Particular examples of osteoinductive factors include TGF-β, IGF-1, IGF-2, BMP-2, BMP-7, GDF-5, parathyroid hormone (PTH), and angiogenic factors. Other osteoinductive factors such as osteocalcin and osteopontin are also likely to be present in DBM preparations as well. There are also likely to be other unnamed or undiscovered osteoinductive factors present in DBM.

Compositions are modified with different modifiers, minerals or polymers, factors, or otherwise to improve fusion capability. Oxysterol compositions can be added to the coating process where collagen is utilized. Polymers may be integrated, for instance in the use of polyurethanes. Peptide integration may also impact coating of the fiber, including use of fibrin PTH (parathyroid hormone). Additionally, BMP growth factors, and alterations or variations including rhBMP-2, may also be added during the coating process prior to lyophilization or for rehydration of the lyophilized substrate. In some embodiments, the mineral coating can allow controlled or prolonged release of the BMP growth factors and, in some embodiments, allow for a reduced amount of BMP growth factors to load the delivery matrix, which can allow for a reduced dosage of BMP growth factors. Such controlled release extends the prior days taken for release when implanted in a body to weeks and/or longer.

In various embodiments, the DBM provided in the methods described in this application is prepared from elongated bone fibers. The bone fibers employed in this application are generally characterized as having relatively high average length to average width ratios, also known as the aspect ratio. In various embodiments, the aspect ratio of the elongated bone fibers is at least from about 50:1 to about at least about 1000:1. Such elongated bone fibers can be readily obtained by any one of several methods, for example, by milling or shaving the surface of an entire bone or relatively large section of bone.

In other embodiments, the length of the fibers can be at least about 3.5 cm and average width from about 20 mm to about 1 cm. In various embodiments, the average length of the elongated fibers can be from about 3.5 cm to about 6.0 cm and the average width from about 20 mm to about 1 cm. In other embodiments, the elongated fibers can have an average length be from about 4.0 cm to about 6.0 cm and an average width from about 20 mm to about 1 cm.

In yet other embodiments, the diameter or average width of the elongated fibers is, for example, not more than about 1.00 cm, not more than 0.5 cm or not more than about 0.01 cm. In still other embodiments, the diameter or average width of the fibers can be from about 0.01 cm to about 0.4 cm or from about 0.02 cm to about 0.3 cm.

In another embodiment, the aspect ratio of the fibers can be from about 50:1 to about 950:1, from about 50:1 to about 750:1, from about 50:1 to about 500:1, from about 50:1 to about 250:1; or from about 50:1 to about 100:1. Fibers according to this disclosure can advantageously have an aspect ratio from about 50:1 to about 1000:1, from about 50:1 to about 950:1, from about 50:1 to about 750:1, from about 50:1 to about 600:1, from about 50:1 to about 350:1, from about 50:1 to about 200:1, from about 50:1 to about 100:1, or from about 50:1 to about 75:1.

To prepare the osteogenic DBM, a quantity of fibers can be combined with a biocompatible carrier to provide a demineralized bone matrix.

DBM typically is dried, for example via lyophilization or solvent drying, to store and maintain the DBM in active condition for implantation. Moreover, each of these processes is thought to reduce the overall surface area structure of bone. As may be appreciated, the structural damage of the exterior surface reduces the overall surface area. Physical alterations to the surface and reduction in surface area can affect cell attachment, mobility, proliferation, and differentiation. The surface's affinity for growth factors and release kinetics of growth factors from the surface may also be altered.

In some embodiments, suitable bone material particles that can be coated with the mineral coating of the current application are available from Medtronic Sofamor Danek, Inc., Memphis, Tenn., in the Grafton™ DBM matrix.

In some embodiments, suitable bone material particles that can be coated with the mineral coating of the current application are available from Medtronic Sofamor Danek, Inc., Memphis, Tenn., in the Magnifuse™ Bone Graft, which is a combination of aseptically processed DBM fibers and surface-demineralized chips, which can be delivered in a resorbable mesh containment system. Magnifuse™ DBM bone graft substitute/bone void filler is osteoinductive and osteoconductive.

In some embodiments, suitable bone material particles that can be coated with the mineral coating of the current application are available from Medtronic Sofamor Danek, Inc., Memphis, Tenn., in the Magnifuse™ II Bone Graft, which is a human based allograft product that has been processed removing the mineral component leaving the organic portion of bone.

In one particular embodiment, suitable the coated bone material fibers of the current application can be with commercially available implantable bone material including, but not limited to, MasterGraft® granules and MasterGraft® mini granules made by Medtronic Sofamor Danek, Inc., Memphis, Tenn., which are biphasic, resorbable, ceramic granules comprising about 15% hydroxyapatite (HA) and about 85% beta-tricalcium phosphate (β-TCP). The granules have a natural, interconnected, porous structure which mimics that natural structure of bone and allows for rapid, homogenous bone ingrowth throughout each granule. Each granule is about 80% porous with an average pore size of about 500 microns and about 125 microns interconnected diameter. MasterGraft® granules have an average diameter of about 1.6 mm to about 3.2 mm. In some embodiments, the granules can have an average diameter of about 0.1 mm to about 0.8 mm. MasterGraft® mini granules have an average diameter of about 0.5 mm to about 1.6 mm. Through a highly porous granular structure and the 15% HA/85% β-TCP chemical composition, MasterGraft® granules and MasterGraft® mini granules facilitate rapid, homogenous osseointegration, which supports the bone healing process by acting as a scaffold over which bone can grow. The porosity of the material provides an excellent basis for vascularization and penetration of associated cells, which support integration of the substitute materials required for healing while preserving the bony architecture and attached gingiva.

In some embodiments, the bone material fibers to be coated or the coated bone material fibers can have macropores having a diameter in a range from about 10, 20, 30, 40, 50, 60, 70, 80, 90, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795 to about 800 μm. In some embodiments, the diameter of each of the micropores can be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 to about 10 microns.

In some embodiments, the bone material fibers to be coated or the coated bone material fibers can have a percent microporosity from about 10 to about 100% or from about 10, 20, 30, 40, 50, 60, 70, 80, 90 to about 100%. In some embodiments, micropores can have a diameter in a range from about 50 μm to about 800 μm. In some embodiments, micropores have a diameter in range from about 10, 20, 30, 40, 50, 60, 70, 80, 90, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795 to about 800 μm.

In some embodiments, the bone material fibers to be coated or already coated can be mixed with a ceramic material in an amount from about 50 to about 98 wt. % and also a polymer in an amount from about 2 to about 50 wt. % based on a total weight of each of the bone material fibers as more particularly described in U.S. patent application Ser. No. 17/018,708 filed Sep. 11, 2020 and assigned to Warsaw Orthopedic, Inc., published as US 2021/0069382, which is incorporated herein by reference in its entirety. The plurality of bone material fibers can each include from about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 to about 98 wt. % ceramic material and from about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 wt. % polymer based on the total weight of each of the bone material fibers. The ceramic material can comprise synthetic ceramic or ceramics including hydroxyapatite and beta-tricalcium phosphate. The ceramic material can be in a powder form. The ceramic material can comprise a calcium to phosphate ratio of between 1.0 to about 2.0. In some embodiments, the calcium to phosphate ratio is between 1.0, 1.1, 1.2, 1.3, 1.4, 1.5, 1.6, 1.7, 1.8, 1.9 to about 2.0.

The ceramic material can be a biphasic calcium phosphate comprising hydroxyapatite in an amount of about 8 to about 22 wt. % and beta-tricalcium phosphate in an amount of about 78 to about 92 wt. %. In some embodiments, the hydroxyapatite is in an amount of about 8, 9, 10, 11, 12, 13, 14, 15, 16, 17, 18, 19, 20, 21 to about 22 wt. % and the beta-tricalcium phosphate in an amount of about 78, 79, 80, 81, 82, 83, 84, 85, 86, 87, 88, 89, 90, 91 to about 92 wt. %.

The porous ceramic materials can comprise hydroxyapatite and beta-tricalcium phosphate. The hydroxyapatite can be in an amount of about 8 to about 22 wt. % based on a total weight of a ceramic granule. The hydroxyapatite can be in a range from about 8, 9, 10, 11, 12, 13, 14, 15, 16, 17, 18, 19, 20, 21 to about 22 wt. %. In some embodiments, the hydroxyapatite can be in a range from about 1 to about 99 wt. %, such as from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99 wt. %.

The beta-tricalcium phosphate can be in an amount of about 78 to about 92 wt. % based on a total weight of a ceramic material. The beta-tricalcium phosphate can be in an amount from about 78, 79, 80, 81, 82, 83, 84, 85, 86, 87, 88, 89, 90, 91 to about 92 wt. %. In some embodiments, the beta-tricalcium phosphate can be in a range from about 1 to about 99 wt. %, such as from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99 wt. %.

The porous ceramic materials can have a calcium to phosphate ratio of between 1.0 to about 2.0. In some embodiments, the calcium to phosphate ratio is between 1.0, 1.1, 1.2, 1.3, 1.4, 1.5, 1.6, 1.7, 1.8, 1.9 to about 2.0.

In some embodiments, the ceramic materials coated with a coating liquid, such as for example, a mineral coating can be embedded in a porous carrier matrix such as cross-linked or non-crosslinked collagen, carboxymethyl cellulose or alginate that can also include identification or surface markers that distinguish them from uncoated ceramic materials.

The polymer component of each of the plurality of bone material fibers can be porcine or bovine collagen, bovine type I collagen, tendon or dermis derived collagen, or a combination thereof.

Each of the plurality of bone material fibers to be coated with a coating liquid or that have been coated with a coating liquid (e.g., mineral coating) can have an average diameter from about 0.1 mm to about 10 mm. For example, each of the bone material fibers can have an average diameter from about 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 to about 10 mm. In some embodiments, the bone material fibers can have a granule size from about 0.09 mm to about 0.8 mm.

Each of the bone material fibers to be coated with a coating liquid or that have been coated with a coating liquid can have an average height and/or length from about 0.05, 0.01 mm to about 10 mm or from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 to about 10 mm.

Nanoceramics are utilized in the development of bone tissue engineered products. Nanohydroxyapatite, non-calcium sulfates, and other nanomaterials may be utilized here alone or as composites with matrix-like polymers, angiogenic factors, growth factors, as well as cells or cell matrices to construct biocompatible implant materials to enhance bone growth. In one embodiment, the ceramic material that can be mixed with each of the bone material fibers to be coated with a coating liquid or that have been coated with a coating liquid can be in the form of porous ceramic materials. Exemplary porous ceramic material suitable for use with the current application include the granules described in U.S. patent application Ser. No. 16/523,259, filed on Jul. 26, 2019 and assigned to Warsaw Orthopedic, Inc., which is published as US 2021/0023258A1 and is incorporated herein by reference in its entirety. The porous ceramic material to be coated with a coating liquid or that have been coated with a coating liquid can have an average diameter from about 50 μm to 1.6 mm. In some embodiments, the average diameter of the granules may be from about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 850, 900, 950, 1000, 1100, 1150, 1200, 1250, 1300, 1350, 1400, 1450, 1500, 1550 to about 1600 μm.

Each of the porous ceramic material to be coated with a coating liquid or that have been coated with a coating liquid can have a Brunauer-Emmett-Teller (BET) surface area from about 0.2 to about 10 m²/g. The BET surface area can be from about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100 m²/g. The increase in surface area further facilitates new bone growth by allowing the fibers to dissolve and release calcium faster than ordinary fibers would that are not coated with the mineral coating liquid.

Each of the porous ceramic material to be coated with a coating liquid or that have been coated with a coating liquid can have a microporosity, and the diameter of the micropores is from about 0.01 to about 10 microns. In some embodiments, the diameter of each of the micropores can be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 to about 10 microns. In some embodiments, the median pore diameter can be about 125 μm and the average pore diameter can be 78 μm.

The plurality of bone material fibers to be coated with a coating liquid or that have been coated with a coating liquid can be made into a variety of shapes after lyophilization or using cryogel applications. The shapes can be cut from a textured or flat shaped sheet of bone material fibers comprising the ceramic material and polymer or can be prepared as individual bone material fibers created in molds.

The bone material fibers to be coated with a coating liquid or that have been coated with a coating liquid are porous, but in some embodiments, the bone material fibers are highly porous. For example, porous bone material fibers can have a porosity from about 10 to about 80% or from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 to about 80%. Highly porous bone material fibers can have a porosity from about 81 to about 99% or from about 81, 82, 83, 84, 85, 86, 87, 88, 89, 90, 91, 92, 93, 94, 95, 96, 97, 98 to about 99%.

The lyophilized coated bone material fibers also have microporosity and microporosity as coated bone material fibers before lyophilization. For example, in some embodiments, the lyophilized coated bone material fibers can have a porosity from about 10 to about 99% or from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99%.

In some embodiments, the lyophilized bone material fiber coated with a nano-apatite surface coating can have an improved surface area of from about 5.31 to about 8.83 m²/g, which represents about an 1106% increase to about an 1804% increase in surface area (an average of 1300%), compared to uncoated bone material fibers. The incubation time of the coating process in accordance with the present application in this embodiment is only about 3 days, which is shorter than the conventional coating method. It is contemplated that the coating methods of the present application can increase the coated surface area of the lyophilized bone material fiber, by for example, an average of 1300%.

In some embodiments, additional or alternative materials may be added to the bone material fibers after they are coated with the coating liquid such as one or more of poly (alpha-hydroxy acids), polyglycolide (PG), polyethylene glycol (PEG) conjugates of poly (alpha-hydroxy acids), polyorthoesters (POE), polyaspirins, collagen, polyphosphagenes, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), methacrylates, PEO-PPO-PAA copolymers, PLGA-PEO-PLGA, PEG-PLG, PLA-PLGA, poloxamer 407, PEG-PLGA-PEG triblock copolymers, POE, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyether ether ketone, polymethylmethacrylate, silicone, hyaluronic acid, or combinations thereof.

In some embodiments, the bone material fibers can comprise at least one biodegradable polymer comprising one or more of collagen, poly(lactide-co-glycolide) (PLGA), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide), carboxymethylcellulose (CMC), alkylene oxide copolymer (AOC) or a combination thereof.

The collagen that can be made into collagen fibers or added to the implantable composition can be from skin, tendon, fascia, ligament, trachea, or organ collagen. In certain embodiments, the collagen is human collagen or another mammalian collagen (e.g., porcine, bovine, or ovine). The collagen can be sourced from any animal. In some embodiments, the collagen can include synthetic self-assembling collagen that can be coated with the nanocoating. While natural collagen fiber sources may create integral substrates with increased efficacy (e.g., reduced BMP2 dosage and controlled release profiles), commercially available collagen and collagen sourced materials can be utilized and deconstructed for use in fibrous form, and reconstructed with the nanocoating to create an integrally nanocoated collagen substrate.

Generally, there are about twenty-eight distinct collagen types that have been identified in vertebrates, including bovine, ovine, porcine, chicken, marine, and human sources. The collagen types are numbered by Roman numerals, and the chains found in each collagen type are identified by Arabic numerals. Detailed descriptions of structure and biological functions of the various types of naturally occurring collagens are generally available in the art.

The collagen may have the same composition as in naturally occurring sources. Examples of sources of collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen may further or alternatively comprise hetero- and homo-trimers of any of the above-recited collagen types. In some embodiments, the collagen comprises hetero- or homo-trimers of human collagen type I, human collagen type II, human collagen type III, or combinations thereof. In some embodiments, the collagen is type I or substantially all is collagen type I, e.g., at least 60%, at least 65%, at least 70%, at least 75%, at least 80%, at least 85%, at least 90%, or at least 95%.

### Coating Liquid

The coating liquid (e.g., mineral coating liquid) that is coated on the bone material fibers imparts increased surface area to that bone material fibers. In some embodiments, the increased surface area increases the hydrophilic properties of the coated bone material fibers and makes the bone implant formed from those coated bone material fibers more hydrophilic and more osteoinductive and osteoconductive allowing the implant to better integrate with the bone defect. Exemplary coating liquid for use with the current application is disclosed in U.S. patent application Ser. No. 13/879,178, published as U.S. Publication No. 2014/0161886, filed Sep. 25, 2009, hereby incorporated by reference in its entirety.

The coating liquid (e.g., mineral coating liquid) is flowable and can contact the bone material fibers in a controlled manner by incubating, submerging, suspending and/or bathing the coating liquid in those bone material fibers. The coating liquid can comprise a mineral component, a polymer component, an organic component a solvent, a buffer, or a combination thereof.

In some embodiments, the coating liquid comprises a calcium-containing mineral, the calcium-containing mineral is at least one of apatite, hydroxyapatite (HAP), α-tricalcium phosphate, β-tricalcium phosphate, amorphous calcium phosphate, dicalcium phosphate, octacalcium phosphate, calcium carbonate, a carbonated-substituted calcium-deficient hydroxyapatite, anorganic bone or combinations thereof. Anorganic bone refers to bone mineral only, with the organic constituents removed.

The calcium-containing mineral can comprise a plurality of layers, e.g., separate layers having distinct dissolution profiles. Under physiological conditions, solubility of calcium phosphate species can adhere to the following solubility trend: amorphous calcium phosphate>dicalcium phosphate>octacalcium phosphate>β-TCP>HAP. A dicalcium phosphate mineral can have a dissolution rate that is more than fifty times higher than that of HAP. Therefore, creation of a matrix with distinct calcium phosphate coatings allows for a broad range of dissolution patterns.

The mineral coating can impart to the bone material fiber's spherical clusters with a plate-like structure or a plate-like structure and a carbonate-substituted, calcium-deficient hydroxyapatite phase structure. As another example, the coating can be an osteoconductive mineral coating and allows osteoclasts and osteoblasts to remodel bone.

The mineral coating useful for coating the bone material fibers can include at least about 1% or at least about 100% porosity including all discrete values included in this range. The mineral coating can also include a pore diameter from about 1 nm to about 3500 nm including all discrete values included in this range. The mineral coating useful in this application can include a ratio of at least about 0.1 Ca/P to about 10 Ca/P including the discrete values included in this range.

As another example, the mineral coating can include an apatite or an amorphous apatite. Apatite can include calcium phosphate, calcium carbonate, calcium fluoride, calcium hydroxide, calcium citrate or a combination thereof.

As another example, a mineral coating can comprise a plurality of discrete mineral islands on the bone material fibers, or the mineral coating can be formed on the entire surface of the bone material fibers. As another example, the mineral coating can comprise a substantially homogeneous mineral coating. In other embodiments, the mineral coatings can be a calcium-deficient carbonate-containing hydroxyapatite.

As another example, the mineral coating can include hydroxyapatite. Calcium-deficient (non-stochiometric) hydroxyapatite, Ca₁₀₋ₓ(PO₄)₆₋ₓ(HPO₄)ₓ(OH)₂₋ₓ (where x is between 0 and 1) has a Ca/P ratio between 1.67 and 1.5. The Ca/P ratio is often used in the discussion of calcium phosphate phases. Stoichiometric apatite Ca₁₀(PO₄)₆(OH)₂ has a Ca/P ratio of 10:6 normally expressed as 1.67. The non-stoichiometric phases have the hydroxyapatite structure with cation vacancies (Ca²⁺) and anion (OH⁻) vacancies. Hydroxyapatite can be predominantly crystalline, but, in some cases, may be present in amorphous forms. The mineral coatings useful in this application can include from at least about 1% to at least about 100% hydroxyapatite, including the discrete values included in this range.

It is known that the hydroxyapatite within the bones of living organisms are very thin plate-like carbonate structures which have an average of 50 nm length, 2-3 nm thickness and a width of 25 nm (see, for example, Heliyon, Volume 6, Issue 4, 2020, Article e03655). In some embodiments, the surface coating on the bone material fibers is designed to have these characteristics.

In some embodiments, the mineral coating can comprise hydroxyapatite, which can be chemically synthesized or synthetic. In some embodiments, the mineral coating can comprise hydroxyapatite, which can be biologically-derived, for example, from animal origin.

In some embodiments, the mineral coating can include octacalcium phosphate. Octacalcium phosphate has a chemical formula of Ca₈H₂(PO₄)₆ 5H₂O or can also be written as Ca₄HO₁₂P₃. Octacalcium phosphate has been shown to be a precursor to hydroxyapatite. Hydrolysis of octacalcium phosphate can create hydroxyapatite. Octacalcium phosphate can be predominantly crystalline, but, in some cases, may be present in amorphous forms. The mineral coating, in some embodiments, can include at least about 1% to at least about 100% octacalcium phosphate including the discrete values included in this range.

In some embodiments, before coating bone material fibers, the bone material fibers are first cleaned in a caustic bath, rinsed, and then contacted (e.g., incubated, submerged, suspended and/or bathed) with the coating liquid (e.g., mineral coating liquid), which can comprise a modified simulated bodily fluid (mSBF).

Ways to make mSBF coating liquid useful for the current application is described in U.S. patent application Ser. No. 15/060,547 filed Mar. 3, 2016, which is assigned to Warsaw Orthopedic, Inc. and published as US 2016/0271296A1, and is described in U.S. patent application Ser. No. 13/879,178 filed Sep. 25, 2009, which is assigned to Warsaw Orthopedic, Inc., and published as US 2014/0161886A1. These entire disclosures are incorporated herein by reference in their entireties and describe how to make a mineral coating having a plate-like nanostructure on a substrate. These patent applications also describe the formation of a plate-like nanostructure comprising nanoparticles having a size range from about 100 to about 500 nanometers.

In some embodiments, the plate-like nanostructure on the coated bone material particle comprises nanoparticles having a size range from about 5, 10, 15, 20, 25, 30, 35, 40, 45, 50, 55, 60, 65, 70. 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 230, 240, 250, 260, 270, 280, 290, 300, 310, 320, 330, 340, 350, 360, 370, 380, 390, 400, 410, 420, 430, 440,450, 460, 470, 480, 490, to about 500 nanometers or less than 1 micron. In some embodiments, the plate-like nanostructure on the coated bone material fiber comprises nanoparticles having a size range from about 5 to about 500 nanometer or up to less than about 1 micron. In some embodiments, the plate-like nanostructure on the coated bone material fiber comprises nanoparticles having a size range from about 50 to about 200 nanometer. In some embodiments, the plate-like nanostructure on the coated bone material fiber comprises nanoparticles having a size range from about 100 to about 200 nanometer. The mSBF provides a calcium and phosphate-rich environment to facilitate crystallization.

In some embodiments, the plate-like nanostructure on the coated bone material fiber comprises nanoparticles having a size range from about 5, 10, 15, 20, 25, 30, 35, 40, 45, 50, 55, 60, 65, 70. 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 230, 240, 250, 260, 270, 280, 290, 300, 310, 320, 330, 340, 350, 360, 370, 380, 390, 400, 410, 420, 430, 440,450, 460, 470, 480, 490, to about 500 nanometers. In some embodiments, the plate-like nanostructure on the coated bone material fiber comprises nanoparticles having a size range from about 5 to about 500 nanometer. In some embodiments, the plate-like nanostructure on the coated bone material fiber comprises nanoparticles having a size range from about 50 to about 200 nanometer. In some embodiments, the plate-like nanostructure on the coated bone material fiber comprises nanoparticles having a size range from about 100 to about 200 nanometer. The mSBF provides a calcium and phosphate-rich environment to facilitate crystallization.

In one embodiment, the bone material fibers are mixed with MasterGraft® granules made by Medtronic Sofamor Danek, Inc., Memphis, Tenn., which are biphasic, resorbable, ceramic granules comprising about 15% hydroxyapatite (HA) and about 85% beta-tricalcium phosphate (β-TCP). The granules have a natural, interconnected, porous structure which mimics that natural structure of bone and allows for rapid, homogenous bone ingrowth throughout each granule. Each granule is about 80% porous with an average pore size of about 500 microns and about 125 microns interconnected diameter.

The MasterGraft® granules can be coated with a mineral coating simultaneously with the bone material fibers or they can be each coated with the mineral coating separately. To coat MasterGraft® granules, they can be suspended in the coating liquid, which can be a mSBF. A recirculating bath is used to maintain physiologic temperature as well as ensure that a controlled rate of interaction between mSBF and the granules and/or fibers occurs throughout the coating process. The stackable meshes allow for an increased density of material within the container while maintaining mSBF flow around the granules and/or fibers. The mSBF can be refreshed every day for example three days, and then rinsed with water and dried. In some embodiments, mSBF is refreshed frequently from about every 1 hour to every 12 hours. In some embodiments, mSBF is refreshed from about every 1, 2, 3, 4, 5, 6, 7, 8, 9, 10, 11, 12, 13, 14, 15, 16, 17, 18, 19, 20, 21, 22, 23 to about 24 hours. In some embodiments, mSBF is refreshed every 2 hours.

Once coating is complete, the meshes allow transfer of the coated bone material fibers directly to an oven for drying. In some embodiments, the coated bone material fibers in a container may be removed through a filter or a mesh before moving to a drying area (e.g., an oven). As a result, the individual fiber are prevented from interacting with each other and disrupting the growth of the nano-apatite plates that are coated on the surface of the fibers. The coating on the surface of the bone material fibers can be a nano sized coating having a nanocoating thickness and/or nanocoating structure.

In some embodiments, the nanocoating thickness ranges from about 1 nm to about less than 1 micron. In some embodiments, the nanocoating thickness ranges from about 1 nm to about 1000 nm. In some embodiments, the thickness ranges from about 50 to about 200 nm. In some embodiments, the thickness ranges from about 40, 45, 50, 55, 60, 65, 70, 75, 80, 85, 90, 95, 100, 105, 110, 115, 120, 125, 130, 135, 140 145, 150, 155, 160,165, 170, 175, 180, 185, 190, 195, 200, 205, 210, 215, to about 220 nm. In some embodiments, an individual coated bone material fibers has a mineral coating having a thickness from about 50 to about 200 nanometers.

In some embodiments, the liquid coating can be a mineral coating (e.g., mSBF) that also contains a polymer and a solvent. The polymer can be acrylic resin, alginate, caprolactone, collagen, chitosan, hyaluronic acid, hydrogel, hydroxybutyric acid, polyanhydride, polycaprolactone (PCL), poly(dimethylglycolic acid), polydioxanone (PDO), polyester, polyethylene, poly(ethylene glycol), poly(glycolide) (PGA), poly(glycolic acid), polyhydroxobutyrate, poly(2-hydroxyethyl-methacrylate), poly-lactide-co-glycolide (PLCG), poly(D,L-lactide-co-glycolide) (PLG), poly(lactide-co-glycolic acid) (PLGA), polylactide (PLA), polylactic acid (PLLA), poly-lactide-co-glycolide (PLCG), poly(methylethylglycolic acid), polymethylmethacrylate, polyphosphazenes, polyphosphoesters, polypropylene, poly(propylene fumarate), polyurethane (PU), or silicone rubber, or combinations or copolymers thereof. In some embodiments, the polymer in the mineral coating includes a ratio of about 1:1 of two polymers or a polymer and co-polymer thereof. In some embodiments, the polymer in the mineral coating has a grain size of about 10 μm to about 500 μm or an average grain size of about 10 μm to about 500 μm.

In some embodiments, a solvent can be used in the mineral coating. A suitable solvent that can be used in the mineral coating, includes, but is not limited to acetic acid, alcohol, aliphatic ether, aniline, chloroform, chlorinated hydrocarbon, aromatic hydrocarbon, aqueous alkali, aqueous solution of cupriethylenediamine, benzene, biphenyl, chlorinated aliphatic hydrocarbon, chlorinated hydrocarbon, chloroform, chlorophenol, chlorobenzene, cyclohexanone, chlorinated hydrocarbon, chloroauric acid, DCM, dimethylformamide (DMF), DMSO, dichlorobiphenyl, dioxane, dilute aqueous sodium hydroxide, 1,2-dichlorobenzene, dichloromethane, DCM, ethanol, ethyl acetate, ethylene carbonate, esters, formic acid, glycols, halogenated hydrocarbons, HFIP, higher aliphatic ester, higher aliphatic ketone, halogenated hydrocarbon, higher aliphatic ester, higher aliphatic ketone, ketone, higher ketone, hydrocarbon, isopropylamine, methyl ethyl ketone, morpholine, methylene chloride, methanol, methyl ethyl ketone, m-Cresol, NMP, phenol, phenylenediamine, sulfuric acid, tetramethylurea, toluene, trifluoroacetic acid, THD, tetramethylurea, tetrahydrofuran (THF), trifluoroacetic acid, trichloroethanol, toluene, trichloroethane, trichloroacetaldehyde hydrate, perfluorokerosene, pyridine, phenyl ether, piperazine, pyridine, water, or xylene, or combinations thereof.

In some embodiments, the solvent is selected from 2 or more of the following solvents: chloroform, acetic acid, formic acid, or a combination of formic acid and acetic acid. In some embodiments, the ratio of the two solvents is 4:1, 3:1, 2:1, or 1:1.

In some embodiments, the percent weight of polymer to volume of solvent is about 1% w/v to about 10% w/v; or about 1% w/v, about 2% w/v, about 3% w/v, about 4% w/v, about 5% w/v, about 6% w/v, about 7% w/v, about 8% w/v, about 9% w/v, or about 10% w/v.

In some embodiments, the coating liquid comprises a calcium-containing mineral and further includes NaCl, KCl, MgCl₂, MgSO₄, NaHCO₃, CaCl₂), KH₂PO₄, or a combination thereof to form the modified simulated body fluid.

In some embodiments, the coating liquid comprises a calcium-containing mineral and further comprises: (i) NaCl has a concentration of about 100 mM to about 200 mM; (ii) KCl has a concentration of about 1 mM to about 8 mM; (iii) MgCl₂ has a concentration of about 0.2 mM to about 5 mM; (iv) MgSO₄ has a concentration of about 0.2 mM to about 5 mM; (v) NaHCO₃ has a concentration of about 1 mM to about 100 mM; (vi) CaCl₂) has a concentration of about 2 mM to about 20 mM; and (vii) KH₂PO₄ has a concentration of about 0.5 mM to about 10 mM to form the mSBF. In some embodiments, the coating liquid further comprises: (i) NaCl has a concentration of about 133 mM to about 148 mM; (ii) KCl has a concentration of about 3.8 mM to about 4.2 mM; (iii) MgCl₂ has a concentration of about 0.95 mM to about 1.05 mM; (iv) MgSO₄ has a concentration of about 0.47 mM to about 0.53 mM; (v) NaHCO₃ has a concentration of about 4 mM to about 4.4 mM; (vi) CaCl₂) has a concentration of about 4.85 mM to about 5.15 mM; and (vii) KH₂PO₄ has a concentration of about 1.94 mM to about 2.1 mM to form the mSBF. In some embodiments of the method, (i) NaCl has a concentration of about 141 mM; (ii) KCl has a concentration of about 4.0 mM; (iii) MgCl₂ has a concentration of about 1.0 mM; (iv) MgSO₄ has a concentration of about 0.5 mM; (v) NaHCO₃ has a concentration of about 4.2 mM; (vi) CaCl₂) has a concentration of about 5 mM; and (vii) KH₂PO₄ has a concentration of about 2.0 mM to form the mSBF.

In some embodiments, the simulated body fluid includes a buffer comprising DPBS, Tris, Tris-HCl, Tris-buffered saline, PBS, or a combination thereof. The buffer can be at a concentration of about 3 mM to about 40 mM. In some embodiments, Tris has a concentration of about 19 to about 21 mM. In some embodiments, Tris has a concentration of about 20.0 mM.

In some embodiments, a pre-condition solution is used in the coating process. The pre-condition solution comprises NaOH having a concentration from about 0.01 to 0.5 M. In some embodiments, NaOH has a concentration less than or equal to 0.5 M.

In yet other embodiments, the pH of mSBF can be varied between about 5.0 and about 6.0 to promote hydroxyapatite formation. Similarly, the pH of mSBF can be varied between about 6.0 and about 6.5 to promote octacalcium phosphate and hydroxyapatite formation. Likewise, the pH of mSBF can be varied between about 6.5 and about 8.0 to promote dicalcium phosphate, octacalcium phosphate, and hydroxyapatite formation. In some embodiments, the coating liquid has a pH of about 6.5 to about 7.5 In some embodiments, the coating liquid has an average pH of 6.8.

In some embodiments, the period of time sufficient to form coated bone material fibers is about 1 day to about 4 days. In some embodiments, incubating the coating liquid with the bone material fibers to form nanostructurally coated bone material fibers can be done for a period of time for at least about 1 day; at least about 2 days; at least about 3 days; or at least about 4 days; or at least about 5 days; or at least about 6 days; or at least about 7 days. In some embodiments, incubating the coating liquid with the bone material fibers to form nanostructurally coated bone material fibers can be done in about 4 days.

In some embodiments, the mineral coating includes (i) about 9% to about 100% hydroxyapatite; (ii) about 90% to about 100% hydroxyapatite; or (iii) about 97% hydroxyapatite. In some embodiments, the mineral coating includes (i) about 0% to about 30% octacalcium phosphate; (ii) about 0% to about 3% octacalcium phosphate; or (iii) about 3% octacalcium phosphate. In some embodiments, the mineral coating includes a porosity of (i) between about 2% and about 100%; or (ii) between about 20% and about 28%. In some embodiments, the mineral coating includes (i) a pore diameter of between about 1 nm and about 3500 nm pore; or (ii) between about 100 nm and about 350 nm pore diameter.

In some embodiments, the bone material fibers to be coated include a pore diameter (i) between about 200 μm and about 525 μm; (ii) between about 25 μm to about 65 μm; or (iii) more than about 50 μm. In some embodiments, the bone material fiber includes a macrochannel length of more than about 100 μm. In some embodiments, the mineral coating includes (i) about 0.1 to about 18 Ca/P, or (ii) about 1.1 to about 1.76 Ca/P (calcium to phosphate ratio). In some embodiments, the mineral coating includes (i) about 1.67 to about 1.76 Ca/P, (ii) about 1.1 to about 1.3 Ca/P, or (iii) about 1.37 to about 1.61 Ca/P.

In some embodiments, the mineral coating liquid includes a crystallinity of (i) about 9% to about 100%; (ii) about 90% to about 100%; or (iii) about 96.5%.

The bone material fibers can be coated with a mineral coating liquid by incubating the bone material fibers with the mineral coating liquid. For example, in some embodiments, the mineral coating liquid, described herein, can be made by incubating the bone material fibers in modified simulated body fluid (mSBF) for four days or more at a pH of about 6.8 to about 7.4 and at a temperature of about 37° C. The mSBF can be refreshed daily. In general, an increase in pH can favor hydroxyapatite growth, while a decrease in pH can favor octacalcium phosphate mineral growth.

In other embodiments, conditions favorable for hydroxyapatite formation can include a pH between about 5.0 and about 8.0 and a calcium concentration multiplied by a phosphate concentration between about 10⁻⁵ and about 10⁻⁸ M. Conditions favorable for octacalcium phosphate formation include a pH between about 6.0 and about 8.0 and a calcium concentration multiplied by a phosphate concentration between about 10⁻⁵ and about 10⁻⁷⁵ M. Furthermore, conditions favorable for dicalcium phosphate dehydrate formation can include a pH between about 6.0 and about 8.0 and a calcium concentration multiplied by a phosphate concentration between about 10⁻⁴ and about 10⁻⁶ M.

In some embodiments, the coating liquid (e.g., mSBF) that is contacting the bone material fibers, is maintained, replaced, replenished, removed, or has components added to it including for example, polymer, solvent, buffer, or a combination thereof. In some embodiments, maintaining the concentration of modified simulated bodily fluid includes replacing, replenishing, removing, or adding to modified simulated body fluid at least one of NaCl, KCl, MgCl₂, MgSO₄, NaHCO₃, CaCl₂), or KH₂PO₄, or a combination thereof. This can be done using the apparatus of FIG. 2, FIG. 4 or FIG. 5.

In some embodiments, the bone material (e.g., DBM particles, DBM fibers, collagen fibers, collagen particles, etc.) can have one or more vitamins disposed in the nanocoating including, but not limited to, a platelet, a protein, a peptide, vitamins A, B, C, D, E, K, or a combination thereof. In some embodiments, the nanocoating can have one or more antibiotics incorporated in the coating that imparts antimicrobial activity to the coated bone material.

In some embodiments, the nanocoating imparts a hydroxyapatite coating to the bone material that stimulates bone cells and enhances the microenvironment for bone healing and integration. The nanocoating of the bone material, in some embodiments, has the nanotopography that reduces bacterial adhesion to the surface of the coating and in this way reduces unwanted bacterial colonization of the bone material, which may reduce implant failure.

In some embodiments, the nanocoating can have proteins (e.g., BMP-2) and other additives disposed in the coating and uniformly integrated therein that allows controlled release of the protein (e.g., BMP-2) from the coated bone material, which allows for a more responsive substrate for bone grafting. Other matrices may include coating of peptides with rhBMP-2 or other BMPs or with Fibrin PTH to facilitate bone growth fusion once the matrix is implanted into the body.

In some embodiments, the coated bone material fibers are dried under conditions sufficient to form a continuous coating, which include heating the coated bone material fiber to about 50° C. to about 200° C., sufficient to soften, melt, or cure the coating on the bone material fiber. It is also understood that in some embodiments, the mineral coating crystalizes in various spots forming various continuous yet separated coated surfaces homogenously throughout the bone material fibers.

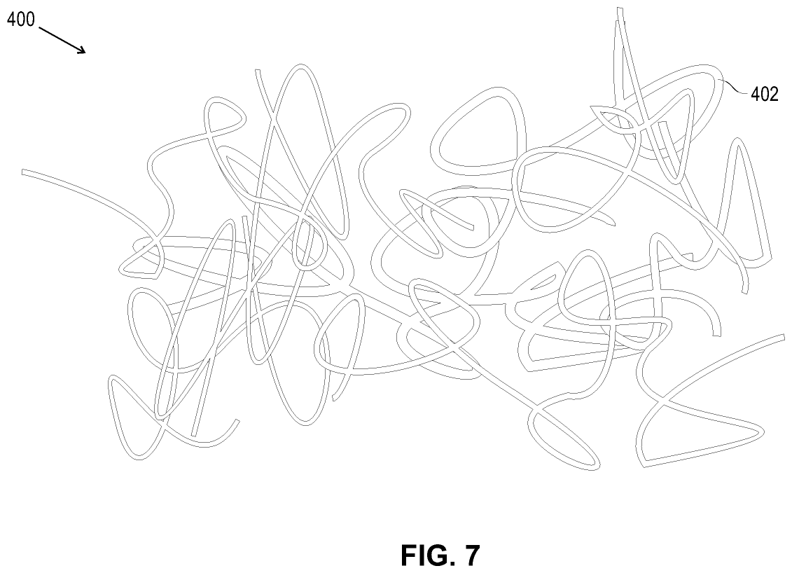

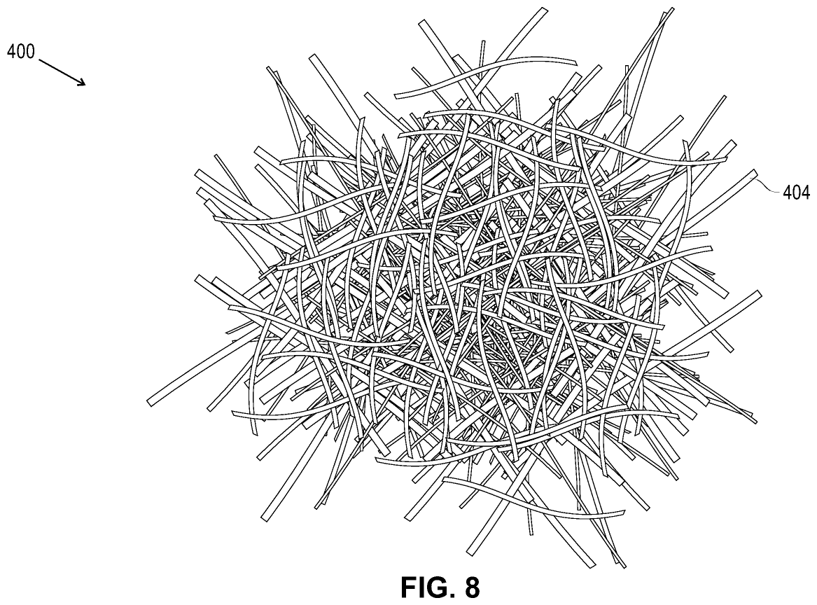

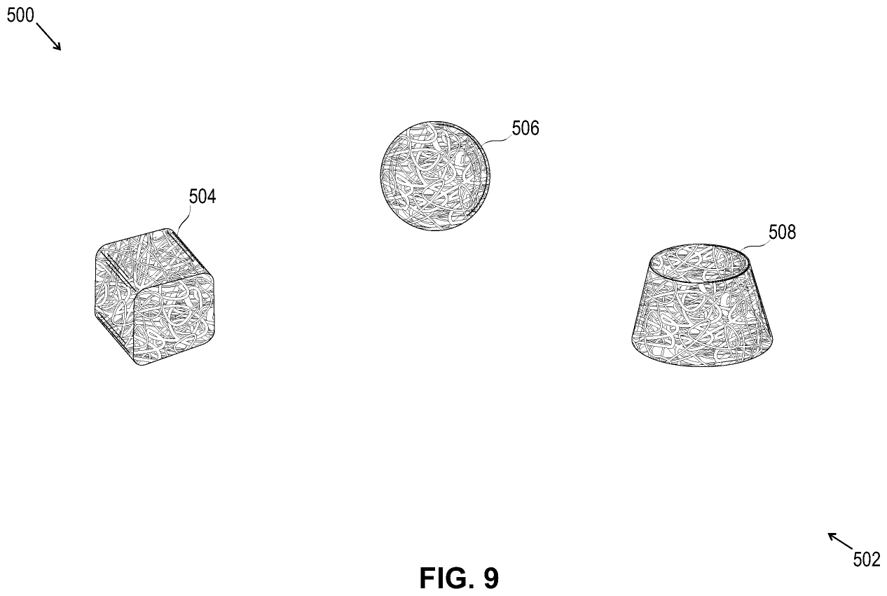

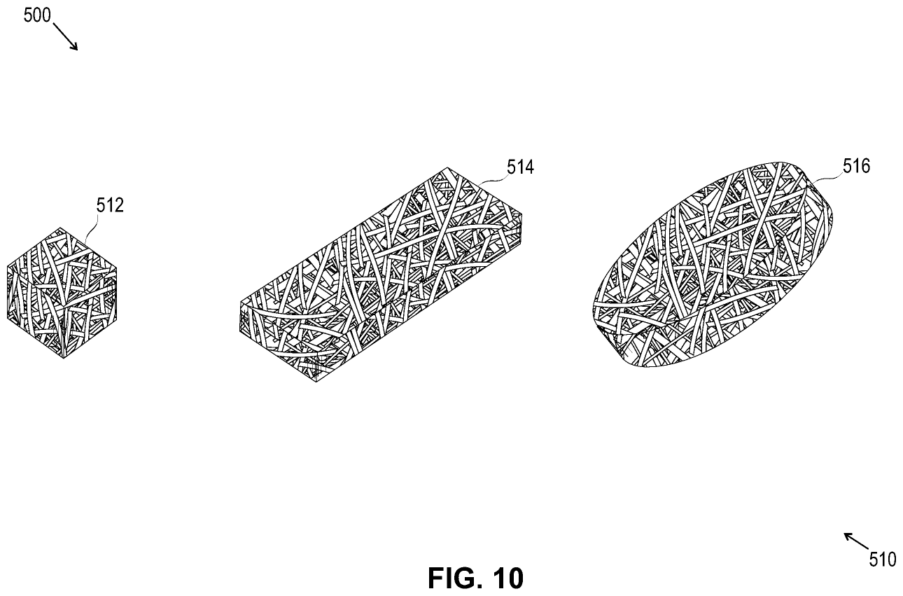

In some embodiments, the method further includes heating the coated bone material fiber for about 1 hour to about 6 hours, sufficient to soften, melt, or cure the coating on the bone material fibers. FIGS. 7 and 8 show embodiments of dried and coated bone material fibers 400 including dried and coated collagen fibers 402 and dried and coated DBM fibers 404. Coated bone material fibers can be dried loosely or by spreading them out or they can be dried in a clump. These dried and coated bone material fibers can be mixed with other commercially available bone implant products as discussed above or they can be collected and molded together. For example, dried coated bone material fibers can be molded into various shapes as molded, dried, coated bone material fibers 500 as illustrated in FIGS. 9 and 10 before and/or after lyophilization. In FIG. 9, the molded, dried, coated collagen fibers 502 comprises various shapes including a cube 504, a sphere 506 and a pyramid 508. In FIG. 10, the molded, dried, coated DBM fibers 510 also comprise various shapes including cube 512, rectangular 514 and disc 516 shapes. It is understood that the dried, coated bone material fibers can be molded into any shape suitable for repairing a bone defect. In some embodiments, lyophilization is conducted while the bone material fibers are still in the mold. In some embodiments, lyophilization is conducted after the bone material fibers are removed from the mold.

In many embodiments, the mineral coating covering the plurality of bone material particle comprises a plate-like nanostructure comprising nanoparticles having a size range from about 100 to about 200 nanometers or up to 1 micron. In some embodiments, the size range of the nanoparticles can be form about 10 to about 100 nanometers.

In many embodiments, the mineral coating covering the plurality of bone material fiber comprises a plate-like nanostructure comprising nanoparticles having a size range from about 100 to about 200 nanometers. In some embodiments, the size range of the nanoparticles can be form about 10 to about 100 nanometers.

The coated bone material fiber shapes and sizes create a high level of surface area, which increases uniform hydration when fluid is administered to the bone material fiber. For example, due to the high level of surface area, fluid will rapidly move into the bone material fiber through wicking. The coated bone material fiber can also be lyophilized.

In some embodiments, the bone material fiber coated with a nano-apatite surface coating can have an improved surface area of from about 5.31 to about 8.83 m²/g, which represents about an 1106% increase to about an 1804% increase in surface area (an average of 1300%), compared to uncoated bone material fibers. The incubation time of the coating process in accordance with the present application in this embodiment can be for example about 3 days, which is shorter than the conventional coating method. The coating methods of the present application also increases the coated surface area of the bone material fiber by an average of 1300%.

In some embodiments, a coated fiber is provided, the coated fiber comprising demineralized bone, collagen or a combination thereof, the coated fiber having a mineral coating thereon, the mineral coating comprising nanostructures having an average size range from about 5 to about 500 nanometers, wherein the coated fiber has a BET surface area from about 0.1 to about 9.5 m²/g.

In some embodiments, there is a coated bone material fiber provided that is made by a process of providing a first mesh having a first set of openings to allow coating liquid and bone material fibers of a select size therethrough and bone material fibers larger than the select size to remain on the first mesh; adding bone material fibers to the first mesh; and contacting the bone material fibers with the coating liquid so as to allow bone material fibers larger than the first set of openings to remain on the first mesh and bone material fibers smaller than the first set of openings and the coating liquid to pass therethrough so as to coat bone material fibers at least on the first mesh.

In some embodiments, a container comprises a beaker, bag, flask, tray, cup, glass, mug, or tumbler capable of holding coating liquid and allows for easy retrieval of coated fibers from the container. In some embodiments, the container comprises an opening configured for adding bone material fibers to be coated, a seal configured to enclose the bone material in the interior of the container, an inlet configured to add a coating liquid to the interior of the container, an outlet configured to remove any excess coating material from the interior of the container, and a sensor configured to sense or detect a parameter within the interior of the container. The parameters that the sensor can detect include, for example, temperature, pressure, weight, pH, humidity, air volume, flow volume, or the sensor can be a camera to visualize the coating environment. In some embodiments, the inlet comprises a liquid input, a gas input, and an optional or spare input. In some embodiments, the outlet comprises a liquid output, a gas output, and an optional or spare output. In some embodiments, the container also comprises a sensor output configured to transfer the measurements collected by the sensor to a computer or a processor. In some embodiments, the container comprises a sampling inlet configured for a user to collect a sample of the bone material, a sample of the coating material, and/or a sample of the mixture of the two to test its contents. Exemplary containers for use with the current application are as disclosed in U.S. patent application Ser. No. 17/522,593, filed Nov. 9, 2021, hereby incorporated by reference in its entirety.

### Methods of Making

In various embodiments, the coated bone material fibers are used to form an implantable bone implant or an implantable composition that can be implanted into the bone defect. In some embodiments, the plurality of coated bone material particles (e.g., bone material fibers) are combined to form a single implant.

In some embodiments, the plurality of coated bone material fibers are mixed with other bone materials to form an implant. The implants formed by individually coated bone material fibers have increased coated surface areas as compared to implants formed by other non-individually coated fibers. Also, each of the plurality of coated bone material fibers has increased surface area compared to a plurality of uncoated bone material fibers. The bone implant with the coated bone material fibers now has increased surface area that is beneficial for hydration of the implant and bone remodeling. In some embodiments, the bone implant includes one or more channels (which are recesses), which not only serve as surface markers but also increase the surface area and hydration characteristics of bone implant. Channels are recesses that can have a macro half-cylindrical shape, or other shapes, for example, a disc shape, a trapezoidal shape or square shape, which can be easily identifiable under visible light. In some embodiments, the implant is a matrix that is in the shape of a disc or in a planar shape.

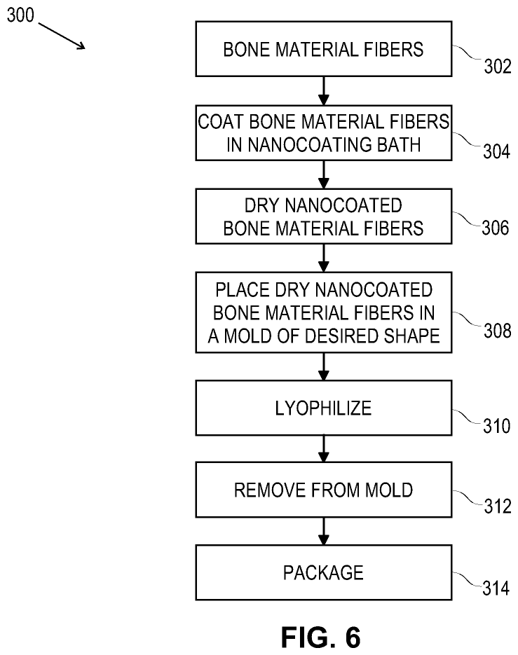

Referring to the process flow diagram of FIG. 6, according to yet another aspect, there is a method of making 300 an implantable bone implant or an implantable composition containing the coated bone material fibers that can be implanted into the bone defect. The method comprises providing bone material fibers 302. In some embodiments, the provided bone material fibers are pre-treated by chemical or physical sterilization, and acid treatment before coating. In some embodiments, the pre-treatment is not required. For example, in some embodiments, collagen fibers are pre-treated before coating. In some embodiments, collagen gel that is used to combined with the bone material fibers are not pre-treated. The bone material fibers are disposed in a nanocoating bath 304 via a container system discussed above, a mesh system discussed above, or a combination thereof. The nanocoated bone material fibers are dried 306 by placing the coated bone material fibers in a dry area or in an oven to accelerate the drying process. The dried, coated bone material fibers are then placed into a mold of desired shape 308. In the process of molding, the bone material fibers are clumped, entangled or packed into the desired shape that can maintain its integrity after removal from the mold. Lyophilization 310 can be conducted on the dried, coated bone material fibers, before, during, or after the molding process. The molded, dried, coated bone material fibers are removed from the mold 312 for further processing, including additional coating, drying, lyophilization or combination with other bone implant products. The bone material fibers, after all desired processes are completed, become the implantable bone implant or implantable composition that is packaged 314 for a provider to use on a patient in need. In some embodiments, the entire process is done aseptically without terminal sterilization. In some embodiments, aseptic process is optional and the end product such as molded, dried, coated bone material fibers are sterilized before reaching an end user such as a provider or a patient. It is understood that the process may be conducted in this specific order or in no particular order to achieve molded, dried, coated and/or lyophilized bone material fibers that become the implantable bone implant or implantable composition. It is also understood that other bone materials suitable to be combined or mixed with the bone material fibers may be coated with the fibers. In some embodiments, the other bone materials are coated separately but molded together with the coated bone material fibers. In some embodiments, the other bone materials are combined or mixed with the coated bone material fibers after the coated bone material fibers are molded. In some embodiments, the bone implant having the coated bone material fibers and other suitable bone material are coated again.

In various embodiments, the nanocoated fibers are homogenously distributed into a formed bone implant or a matrix thereby forming a bone grafting material or implant having nanocoating integrated in the interior and/or exterior of the matrix. The increased surface area of the nanocoating enhances the functional characteristic of the bone implant to improve functionality of the coating, as the increased surface is not limited to just the outer/exterior surface of the entire bone implant, but each individual fiber in the implant.

In some embodiments, each individual bone material fiber can be uniformly exposed to a mineral coating liquid, however, if the bone material fibers are in bulk, or a cluster or entangled, the exposure to mineral coating liquid may not be uniform and may result in a non-uniform coating.

In various embodiments, the plurality of coated bone material fibers may be mixed with already existing bone implant products such as bone strips, putty and pastes. Such bone implant products may include, but are not limited to, MasterGraft® Strips produced by Medtronic Sofamor Danek, Inc., Memphis, Tenn.; MasterGraft® Putty produced by Medtronic Sofamor Danek, Inc., Memphis, Tenn., and/or Matrix EXT compression resistant products produced by Medtronic Sofamor Danek, Inc., Memphis, Tenn. Other bone implant products include porous ceramic granules as described in U.S. patent application Ser. No. 16/523,204, filed Jul. 26, 2019, and published as U.S. Publication No. 2021/0024430, hereby incorporated by reference in its entirety; as also described in U.S. patent application Ser. No. 16/523,237, published as U.S. Pat. No. 11,311,647, filed Jul. 26, 2019, hereby incorporated by reference in its entirety; and as also described in U.S. patent application Ser. No. 16/523,259, filed Jul. 26, 2019, published as U.S. Publication No. 2021/0023258, hereby incorporated by reference in its entirety.

In various embodiments, a method of treating a bone defect is provided. The method comprises implanting a bone implant into the defect, the bone implant comprising a bone material fiber comprising hydroxyapatite in an amount of about 8 to about 22 wt. % and beta-tricalcium phosphate in an amount of about 78 to about 92 wt. %, the bone material fiber having micropores having an average diameter of about 50 μm to about 800 μm, and each bone material fiber having an average fiber size of about 0.1 mm to 2.0 mm, the bone material fiber having a mineral coating thereon, the mineral coating comprising nanostructures having an average size range from about 5 to about 500 nanometers, wherein the porous ceramic granule has a BET surface area from about 0.4 to about 9.5 m²/g.

In some embodiments, the coated bone material fiber can have a BET surface area from about 0.1, 0.2, 0.3, 0.4, 0.5, 0.6, 0.7, 0.8, 0.9, 1.0, 1.5, 2.0, 2.5, 3.0, 3.5, 4.0, 4.5. 5.0, 5.5, 6.0, 6.5, 7.0, 7.5, 8.0, 8.5, 9.0 to about 9.5 m²/g. In some embodiments, the bone material fibers before coating can have a BET surface area from about 0.1, 0.2, 0.3, 0.4 to about 0.5 m²/g.

In some embodiments, the bone implant containing the mineral coating can be hydrated with fluid comprising bone marrow aspirate, saline, sterile water, blood for injection, phosphate buffered saline, dextrose, Ringer's lactated solution, or a combination thereof before, during or after the bone implant is implanted into the bone defect. The ratio of fluid to the coated bone material fiber can be from about 0.5:1 to about 3:1. In some embodiments, the ratio of fluid to the coated bone material fibers can be from about 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1 to about 3:1.

In some embodiments, the hydrated coated bone material particles (e.g., fibers) can have a flowable viscosity starting from about 50 Pascal-second (Pa-s), 100 Pa-s, 150 Pa-s, 200 Pa-s, 250 Pa-s, to about 300 Pa-s and reaches a higher viscosity from about 500 Pa-s, 750 Pa-s, 1000 Pa-s, 1,500 Pa-s, 2,000 Pa-s, 2,500 Pa-s to about 3,000 Pa-s. In some embodiments, the hydrated bone material particles (e.g., fibers) can have a flowable viscosity starting from about 50 Pa-s to about 3,000 Pa-s and reach a higher viscosity from about 3,000 Pa-s to about 300,000 Pa-s.

The coated bone material particles (e.g., fibers) can have a certain density when hydrated. For example, when the coated bone material particles (e.g., fibers) are hydrated, the density can be from about 1.2 to about 2.0 g/cc or from about 1.4 to about 1.6 g/cc. In some embodiments, the hydrated bone material particles (e.g., fibers) can have a density from about 1.2, 1.3, 1.4, 1.5, 1.6, 1.7, 1.8, 1.9 to about 2.0 g/cc.

The coated bone material particles (e.g., fibers), once hydrated and placed into an implant can have a modulus of elasticity from about 2 MPa to about 12 MPa, such as from about 2, 3, 4, 5, 6, 7, 8, 9, 10, 11 to about 12 MPa.

The coated bone material particles (e.g., fibers) can be combined and formed into an implant and/or lyophilized. The implant can be hydrated with liquid to the desired consistency such as a gel, paste, or putty (e.g., a granular putty). In some embodiments, the liquid can be removed or the implant dehydrated to achieve the desired consistency. For example, an injectable implant that is in a liquid state can have liquid removed from it and the implant can be formed into a less viscous gel, putty, or paste.

Although the invention has been described with reference to preferred embodiments, persons skilled in the art will recognize that changes may be made in form and detail without departing from the spirit and scope of the invention.

What is claimed is:

1. An implant comprising a plurality of coated bone material particles, each of the plurality of coated bone material particles having a mineral coating thereon, the mineral coating having nanostructures and the plurality of coated mineral particles distributed throughout the implant.

2. The implant of claim 1, wherein (i) each of said coated bone material particles comprises demineralized bone, cellular bone material, collagen, synthetic bone, or a combination thereof; or (ii) each of said coated bone material particles are homogenously distributed throughout the implant.

3. The implant of claim 1, wherein the plurality of coated bone material particles are fibers disposed within the implant.

4. The implant of claim 3, wherein the mineral coating comprises a hydroxyapatite, a non-calcium phosphate, a polymer, or a protein.

5. The implant of claim 4, wherein (i) the hydroxyapatite is a nanohydroxyapatite coating; (ii) the hydroxyapatite is synthetic; or (iii) the hydroxyapatite is biologically-derived.

6. The implant of claim 4, wherein the implant is in the form of a matrix, which is a gel, a paste, a putty, a granular putty, or in a shape of a planar structure or disc.

7. The implant of claim 5, wherein the implant is freeze-dried or lyophilized to form a matrix.

8. The implant of claim 6, wherein the matrix is hydratable.

9. The implant of claim 7, wherein the implant is dehydrated.

10. The implant of claim 3, wherein the mineral coating comprises (i) a bone morphogenetic protein, fibrin, a hormone, an antimicrobial, a vitamin, a platelet, or a combination thereof; or (ii) a peptide, a protein, a human growth factor, or a combination thereof.

11. The implant of claim 4, wherein the mineral coating reduces bacterial adhesion to a surface of the coated bone material particle.

12. The implant of claim 1, wherein the mineral coating comprises a calcium-containing mineral, the calcium-containing mineral is at least one of apatite, hydroxyapatite, α-tricalcium phosphate, β-tricalcium phosphate, amorphous calcium phosphate, dicalcium phosphate, octacalcium phosphate, calcium carbonate, or a carbonated-substituted calcium-deficient hydroxyapatite, anorganic bone, or a combination thereof and the mineral coating is a nanocoating integrated with nanostructures.

13. The implant of claim 3, wherein the fibers comprise fibrous connective tissue or fibrous bone tissue.

14. A coated bone material particle, the coated bone material particle having a mineral coating thereon, the mineral coating having nanostructures and the mineral coating comprising calcium.

15. The coated bone material particle of claim 14, wherein (i) the coated bone material particle comprises demineralized bone, collagen or a combination thereof in fiber form; (ii) a plurality of coated bone material particles are combined into an implant; or (iii) a plurality of coated bone material particles are combined into the implant and lyophilized.

16. A method of coating bone material fibers, the method comprising:
providing a container configured to receive a mineral coating liquid and a plurality of bone material fibers therein;
adding the plurality of bone material fibers to the container; and
contacting the plurality of bone material fibers with the mineral coating liquid in the container so as to coat each individual bone material fiber with the mineral coating liquid to form a plurality of coated bone material fibers.

17. The method of claim 16, wherein the plurality of bone material fibers added to the container comprise demineralized bone fibers, collagen fibers or a combination thereof.

18. The method of claim 16, wherein the mineral coating liquid comprises a calcium-containing mineral, the calcium-containing mineral comprising at least one of apatite, hydroxyapatite, α-tricalcium phosphate, β-tricalcium phosphate, amorphous calcium phosphate, dicalcium phosphate, octacalcium phosphate, calcium carbonate, or a carbonated-substituted calcium-deficient hydroxyapatite, anorganic bone, or a combination thereof.

19. The method of claim 16, wherein the mineral coating liquid is applied to form a thickness of less than a micron on each individually coated bone material fiber.

20. The method of claim 16, wherein the mineral coating liquid is uniformly disposed on a surface of each individually coated bone material fiber.
